(19) United States
(12) Patent Application Publication
(10) Pub. No.: US 2023/0390052 A1
(43) Pub. Date: Dec. 7, 2023

(54) PROSTHETIC VALVE SYSTEMS, COMPONENTS, AND METHODS

(71) Applicant: EDWARDS LIFESCIENCES CORPORATION, Irvine, CA (US)

(72) Inventors: Ikechukwu Uzoma Okafor, Irvine, CA (US); Matthew A. Peterson, Costa Mesa, CA (US); Eva Adriana de la Cruz Romito, Irvine, CA (US)

(21) Appl. No.: 18/227,235

(22) Filed: Jul. 27, 2023

Related U.S. Application Data
(63) Continuation of application No. PCT/US2022/015476, filed on Feb. 7, 2022.
(60) Provisional application No. 63/148,058, filed on Feb. 10, 2021.

(51) Int. Cl. A61F 2/24

(52) U.S. Cl. CPC A61F 2/2418; A61F 2220/0091; A61F 2220/0008; A61F 2002/0081

(57) ABSTRACT

Apparatuses, systems, and methods for prosthetic valves. Embodiments of prosthetic valves may be directed to sealing flow at an implantation site, which may comprise a native valve. Embodiments may be configured to conform to a shape of a native valve, which may be a non-circular shape. Embodiments may include a frame having portions coupled together by one or more rotation joints. Embodiments may include a crescent shaped frame. Embodiments may include a construction of a frame having integral portions. Embodiments may include a portion of a prosthetic valve having a micropattern applied to the prosthetic valve. One or more microbeads may be utilized in embodiments. A construction of a prosthetic valve utilizing a support ring may be provided.

### CROSS-REFERENCE TO RELATED APPLICATIONS

This application is a continuation of International Application No. PCT/US2022/015476, filed Feb. 7, 2022, which designates the United States and was published in English by the International Bureau on Aug. 18, 2022 as WO2022/173692, which claims the benefit of U.S. Provisional Application No. 63/148,058, filed Feb. 10, 2021, the entire contents of each of which are hereby incorporated by reference.

### BACKGROUND

### Field

Certain embodiments disclosed herein relate generally to implants, including prosthetic valves for implantation.

### Background

Human heart valves, which include the aortic, pulmonary, mitral and tricuspid valves, function essentially as one-way valves operating in synchronization with the pumping heart. The valves allow blood to flow downstream, but block blood from flowing upstream. Diseased heart valves exhibit impairments such as narrowing of the valve or regurgitation, which inhibit the valves' ability to control blood flow. Such impairments reduce the heart's blood-pumping efficiency and can be a debilitating and life threatening condition. For example, valve insufficiency can lead to conditions such as heart hypertrophy and dilation of the ventricle. Thus, extensive efforts have been made to develop methods and apparatuses to repair or replace impaired heart valves.

Prostheses exist to correct problems associated with impaired heart valves. For example, mechanical and tissue-based heart valve prostheses can be used to replace impaired native heart valves. More recently, substantial effort has been dedicated to developing replacement heart valves, particularly tissue-based replacement heart valves that can be delivered with less trauma to the patient than through open heart surgery. Replacement valves are being designed to be delivered through minimally invasive procedures and even percutaneous procedures. Such replacement valves often include a tissue-based valve body that is connected to an expandable frame that is then delivered to the native valve's annulus.

These replacement valves are typically “one-way valves” that allow blood to flow in only one direction. However, a problem occurs when blood leaks around the outside of the prosthesis. For example, in the context of replacement heart valves, paravalvular leakage (PVL) has proven to be particularly challenging. An additional challenge relates to the ability of such prostheses to be secured relative to intralumenal tissue, e.g., tissue within any body lumen or cavity, in an atraumatic manner.

### SUMMARY

Embodiments of prosthetic valves may be directed to improvements in sealing flow at an implantation site, which may comprise a native valve. Embodiments may be configured to conform to a shape of a native valve, which may be a non-circular shape. Such native valves may include native valve annuli having ovoid shapes and/or shapes that include one or more recesses.

Embodiments may further include improvements in the construction of prosthetic valves, including a configuration of components of prosthetic valves, which may comprise prosthetic valve leaflets.

Embodiments as disclosed herein may include a prosthetic valve configured to be deployed to a native valve. The prosthetic valve may include one or more prosthetic valve leaflets. The prosthetic valve may include a frame coupled to the one or more prosthetic valve leaflets and including a first portion and a second portion, and at least one rotation joint coupling the first portion to the second portion and allowing the first portion to rotate relative to the second portion.

Implementations of the embodiments may include one or more of the following. The at least one rotation joint may include one or more of a suture joint, a t-bracket, a ball and socket joint, an overmolding, or a hinge. The suture joint may include a shear-lashing knot. The frame may comprise an outer frame spaced from an inner frame, the inner frame configured to support the one or more prosthetic valve leaflets and the outer frame configured to conform to a shape of an annulus of the native valve. The outer frame may surround the inner frame. The outer frame may have a bulbous shape. The inner frame may have a circular shape. A skirt may be coupled to the outer frame and configured to seal a portion of the annulus. A proximal portion of the outer frame may be coupled to a proximal portion of the inner frame, and the outer frame is spaced from the inner frame at a gap. The first portion may include at least one strut cell, and the second portion includes at least one strut cell. The first portion may comprise a column of the frame, and the second portion comprises an adjacent column of the frame. The frame may surround a central axis of the prosthetic valve, and the first portion is configured to rotate relative to the second portion in a plane extending transverse to the central axis. The first portion may be configured to rotate relative to the second portion to vary a shape of an outer surface of the frame. One or more distal anchors may be configured to extend around one or more leaflets of the native valve to anchor the prosthetic valve to the native valve. The prosthetic valve may be configured to be deployed to a mitral valve or a tricuspid valve.

Embodiments as disclosed herein may include a method. The method may include deploying a prosthetic valve to a native valve, the prosthetic valve including one or more prosthetic valve leaflets, and a frame coupled to the one or more prosthetic valve leaflets and including a first portion and a second portion, and at least one rotation joint coupling the first portion to the second portion and allowing the first portion to rotate relative to the second portion.

Implementations of the embodiments may include one or more of the following. The method may include allowing the first portion to rotate relative to the second portion in response to a shape of an annulus of the native valve. The method may include allowing the frame to conform to the shape of the annulus of the native valve. The method may include deploying the prosthetic valve to a mitral valve or a tricuspid valve.

Embodiments as disclosed herein may include a prosthetic valve configured to be deployed to a native valve. The prosthetic valve may include one or more prosthetic valve leaflets. The prosthetic valve may include an outer frame having a crescent shape with two ends circumferentially spaced from each other. The prosthetic valve may include an inner frame supporting the one or more prosthetic valve leaflets and surrounded by at least a portion of the outer frame and coupled to each of the two ends of the outer frame.

Implementations of the embodiments may include one or more of the following. A portion of the inner frame positioned between the two ends of the outer frame may form an outermost frame surface of the prosthetic valve. A skirt may be coupled to the outer frame and the portion of the inner frame positioned between the two ends of the outer frame. The skirt may form an outer surface of the prosthetic valve. A center of the inner frame may be offset from a center of the outer frame. The outer frame and the inner frame may together form a “D” shape of the prosthetic valve. The outer frame may have a diameter and the inner frame may have a diameter that is smaller than the diameter of the outer frame. A proximal portion of the outer frame may be coupled to a proximal portion of the inner frame. Connectors may join the two ends of the outer frame to the inner frame. At least one of the connectors may comprise a rotation joint. The outer frame may have a circular shape or an ovoid shape. The inner frame may have a circular shape. The outer frame may be configured to move to conform to a shape of an annulus of the native valve. One or more distal anchors may be configured to extend around one or more leaflets of the native valve to anchor the prosthetic valve to the native valve. The prosthetic valve may be configured to be deployed to a mitral valve or a tricuspid valve.

Embodiments as disclosed herein may include a method. The method may include deploying a prosthetic valve to a native valve. The prosthetic valve may include one or more prosthetic valve leaflets, an outer frame having a crescent shape with two ends circumferentially spaced from each other, and an inner frame supporting the one or more prosthetic valve leaflets and surrounded by at least a portion of the outer frame and coupled to each of the two ends of the outer frame.

Implementations of the embodiments may include one or more of the following. The method may include deploying the prosthetic valve to a mitral valve or a tricuspid valve.

Embodiments as disclosed herein may include a prosthetic valve configured to be deployed to a native valve. The prosthetic valve may include one or more prosthetic valve leaflets. The prosthetic valve may include an inner frame supporting the one or more prosthetic valve leaflets. The prosthetic valve may include a sealing body configured to contact a portion of an annulus of the native valve, the sealing body including a skirt and an outer frame having a plurality of elongate struts, each of the plurality of elongate struts having a proximal portion that is integral with the inner frame and a distal portion that is spaced from the inner frame.

Implementations of the embodiments may include one or more of the following. Each of the plurality of elongate struts may be formed integral with the inner frame. Each of the plurality of elongate struts may be deflected away from the inner frame. The inner frame and the plurality of elongate struts may be formed from a unitary piece of material. The distal portions of each of the plurality of elongate struts may be cut away from the inner frame. The inner frame may include a plurality of struts joined at junctures, and the proximal portion of each of the plurality of elongate struts is integral with one of the junctures. One of the junctures may include one or more of a suture joint, a t-bracket, a ball and socket joint, an overmolding, or a hinge. One of the junctures includes a rotation joint configured to allow a first portion of the inner frame to rotate relative to a second portion of the inner frame. Spaces may be positioned between the plurality of struts. The plurality of struts may form strut cells. Each of the plurality of elongate struts may extend radially outward from the inner frame. The inner frame may include distal anchors configured to extend around one or more leaflets of the native valve to anchor the prosthetic valve to the native valve. The sealing body may further comprise a compressible material positioned between the skirt and the outer frame. The compressible material may include foam. The prosthetic valve may be configured to be deployed to a mitral valve or a tricuspid valve.

Embodiments as disclosed herein may include a method. The method may include deploying a prosthetic valve to a native valve. The prosthetic valve may include one or more prosthetic valve leaflets, an inner frame supporting the one or more prosthetic valve leaflets, and a sealing body configured to contact a portion of an annulus of the native valve, the sealing body including a skirt and an outer frame having a plurality of elongate struts, each of the plurality of elongate struts having a proximal portion that is integral with the inner frame and a distal portion that is spaced from the inner frame.

Implementations of the embodiments may include one or more of the following. The method may include deploying the prosthetic valve to a mitral valve or a tricuspid valve.

Embodiments as disclosed herein may include a method of forming at least a portion of a prosthetic valve configured to be deployed to a native valve. The method may include deflecting a distal portion of each of a plurality of elongate struts of a sealing body away from an inner frame for supporting a plurality of prosthetic valve leaflets, a proximal portion of each of the plurality of elongate struts being formed integral with the inner frame. The method may include providing a skirt on the plurality of elongate struts when the plurality of elongate struts are deflected away from the inner frame.

Implementations of the embodiments may include one or more of the following. The method may include forming the inner frame and the plurality of elongate struts from a unitary piece of material. The unitary piece of material may have a cylindrical shape. Forming the inner frame and the plurality of elongate struts from the unitary piece of material may include cutting the inner frame and the plurality of elongate struts from the unitary piece of material. The method may include pulling the distal portion of each of the plurality of elongate struts away from adjacent struts of the inner frame. The method may include pulling the distal portion of each of the plurality of elongate struts includes forming a gap between the adjacent struts of the inner frame. The method may include closing the gap with a connector. The method may include coupling one or more prosthetic valve leaflets to the inner frame.

Embodiments as disclosed herein may include an apparatus. The apparatus may comprise a prosthetic valve configured to be deployed to a native valve of a body, the prosthetic valve including one or more prosthetic valve leaflets and including a micropattern applied to at least a portion of the prosthetic valve.

Implementations of the embodiments may include one or more of the following. The micropattern may be applied to at least one of the one or more prosthetic valve leaflets. The at least one of the one or more prosthetic valve leaflets may include a first portion and a second portion, the first portion having a first configuration of the micropattern and the second portion having a second configuration of the micropattern that is different than the first configuration. The micropattern may be configured to reduce the formation of a thrombus on the at least one of the one or more prosthetic valve leaflets. The micropattern may be configured to enhance biocompatibility of the at least one of the one or more prosthetic valve leaflets with a patient's body. The micropattern may be configured to alter fluid flow through the prosthetic valve. The one or more prosthetic valve leaflets may include a plurality of the prosthetic valve leaflets, and the micropattern is configured to improve coaptation between the plurality of prosthetic valve leaflets. The prosthetic valve may include a frame configured to support the at least one of the one or more prosthetic valve leaflets within the native valve, and the micropattern is configured to improve coupling of the at least one of the one or more prosthetic valve leaflets to the frame. The micropattern may be laser milled on the at least one of the one or more prosthetic valve leaflets. At least a portion of the prosthetic valve may comprise a fabric, and wherein the micropattern is applied to the fabric. The prosthetic valve may include a sealing skirt, and the micropattern is applied to the sealing skirt. The micropattern may include one or more of a checkered pattern or a sharklet pattern. The micropattern may be on a micrometer length scale. The prosthetic valve may include one or more distal anchors configured to extend around one or more leaflets of the native valve to anchor the prosthetic valve to the native valve. The prosthetic valve may be configured to be deployed to a mitral valve or a tricuspid valve.

Embodiments as disclosed herein may include a method. The method may include deploying a prosthetic valve to a native valve. The prosthetic valve may include one or more prosthetic valve leaflets and a micropattern applied to at least a portion of the prosthetic valve.

Implementations of the embodiments may include one or more of the following. The method may include deploying the prosthetic valve to a mitral valve or a tricuspid valve.

Embodiments as disclosed herein may include a method. The method may include altering a surface roughness of at least a portion of a prosthetic valve, the prosthetic valve including one or more prosthetic valve leaflets and configured to be deployed to a native valve.

Implementations of the embodiments may include one or more of the following. Coupling the at least one of the one or more prosthetic valve leaflets to a frame configured to support the one or more prosthetic valve leaflets within the native valve. Altering the surface roughness may include applying a micropattern to the at least one of the one or more prosthetic valve leaflets. Applying the micropattern may include laser milling the micropattern to the at least one of the one or more prosthetic valve leaflets. Altering the surface roughness may include smoothing a surface of the at least one of the one or more prosthetic valve leaflets. At least a portion of the prosthetic valve may comprise a fabric and altering the surface roughness may include applying a micropattern to the fabric. The prosthetic valve may include a sealing skirt and altering the surface roughness may include applying a micropattern to the sealing skirt. At least a portion of the prosthetic valve may comprise a fabric and altering the surface roughness may include smoothing a surface of the fabric.

Embodiments as disclosed herein may include an apparatus. The apparatus may comprise a prosthetic valve configured to be deployed to a native valve of a body, the prosthetic valve including one or more prosthetic valve leaflets and including one or more microbeads for emitting a substance into the body.

Implementations of the embodiments may include one or more of the following. The prosthetic valve may include a sealing body configured to contact a portion of an annulus of the native valve, and the one or more microbeads are positioned on the sealing body. The prosthetic valve may include a fabric, and the one or more microbeads are coupled to the fabric. The prosthetic valve may include a frame and a skirt coupled to the frame, and the one or more microbeads are coupled to the skirt. The skirt may comprise a sealing skirt. The prosthetic valve may include one or more anchors, and the one or more microbeads are positioned on the one or more anchors. The one or more anchors may comprise one or more distal anchors each configured to hook around a leaflet of the native valve. The one or more microbeads may be positioned on the one or more prosthetic valve leaflets. The prosthetic valve may be configured to be deployed to an aortic valve of the body. The prosthetic valve may be configured to be deployed to a mitral valve or a tricuspid valve of the body. The substance may comprise a drug. The drug may comprise an anti-thrombus drug. The drug may be configured to reduce the formation of a thrombus on the at least one of the one or more prosthetic valve leaflets. The one or more microbeads may be for emitting the substance into a bloodstream of the body. The one or more microbeads may be configured to diffuse to emit the substance into the body.

Embodiments as disclosed herein may include a method. The method may include deploying a prosthetic valve to a native valve of a body, the prosthetic valve including one or more prosthetic valve leaflets and including one or more microbeads for emitting a substance into the body.

Implementations of the embodiments may include one or more of the following. The method may include deploying the prosthetic valve to a mitral valve or a tricuspid valve.

Embodiments as disclosed herein may include a prosthetic valve configured to be deployed to a native valve. The prosthetic valve may include a support ring configured to extend about an annulus of the native valve. The prosthetic valve may include one or more prosthetic valve leaflets coupled to the support ring. The prosthetic valve may include one or more anchors configured to anchor the support ring to the native valve.

Implementations of the embodiments may include one or more of the following. The support ring may be flexible. The support ring may be configured to be expanded from an undeployed configuration to a deployed configuration. The support ring may include a first end portion and a second end portion configured to slide relative to the first end portion to allow the support ring to move. The second end portion may be configured to slide relative to the first end portion to vary a size of a diameter of the support ring. A spring may be configured to bias the first end portion relative to the second end portion. The second end portion may be configured to automatically slide relative to the first end portion in response to a variation in a diameter of the annulus. Each of the one or more prosthetic valve leaflets may include a first end portion coupled to the support ring and a second end portion extending distally from the first end portion. The first end portion may couple to a sheath configured to extend over at least a portion of the support ring. The second end portion may be configured to extend from the first end portion in a direction towards a ventricle. The one or more prosthetic valve leaflets may include at least two prosthetic valve leaflets circumferentially spaced from each other. Each of the one or more prosthetic valve leaflets may be configured to contact and overlay at least a portion of a native valve leaflet. The one or more anchors may each be configured to penetrate into tissue. The one or more anchors may each comprise a screw. The prosthetic valve may be configured to be deployed to a mitral valve or a tricuspid valve.

Embodiments as disclosed herein may include a method. The method may include deploying a prosthetic valve to a native valve. The prosthetic valve may include a support ring configured to extend about an annulus of the native valve, one or more prosthetic valve leaflets coupled to the support ring, and one or more anchors configured to anchor the support ring to the native valve.

Implementations of the embodiments may include one or more of the following. The method may include deploying the prosthetic valve to a mitral valve or a tricuspid valve.

Any of the features of an embodiment disclosed herein, is applicable to all other aspects and embodiments identified herein. Moreover, any of the features of an embodiment of the various embodiments, is independently combinable, partly or wholly with other embodiments described herein in any way, e.g., one, two, or three or more embodiments may be combinable in whole or in part. Further, any of the features of an embodiment may be made optional to other aspects or embodiments. Any embodiment of a method can be performed by a system or apparatus of another embodiment, and any embodiment of a system or apparatus can be configured to perform a method of another embodiment.

### BRIEF DESCRIPTION OF THE DRAWINGS

Features and advantages of the systems, apparatuses, and methods as disclosed herein will become appreciated as the same become better understood with reference to the specification, claims, and appended drawings wherein:

FIG. 1 illustrates an upper perspective view of a prosthetic valve according to embodiments of the present disclosure.

FIG. 2 illustrates a side perspective view of the prosthetic valve shown in FIG. 1.

FIG. 3 illustrates a lower perspective view of the prosthetic valve shown in FIG. 1.

FIG. 4 illustrates a side view of an inner frame of the prosthetic valve shown in FIG. 1.

FIG. 5 illustrates a cross sectional schematic view of the prosthetic valve shown in FIG. 1.

FIG. 6 illustrates a pattern of a portion of an outer frame of a prosthetic valve.

FIG. 7 illustrates a close up view of a rotation joint of the outer frame shown in FIG. 6.

FIG. 8 illustrates a top schematic view of an outer frame shown in FIG. 6 and an inner frame.

FIG. 9 illustrates a schematic view of a delivery apparatus passing into a portion of a patient's body according to an embodiment of the present disclosure.

FIG. 10 illustrates a side schematic view of a prosthetic implant in position to be deployed to a native valve.

FIG. 11 illustrates a side schematic view of a prosthetic implant being deployed to a native valve.

FIG. 12 illustrates a schematic view of a prosthetic implant deployed to a native mitral valve.

FIG. 13 illustrates a top schematic view of the outer frame shown in FIG. 8 moved from the position shown in FIG. 8.

FIG. 14 illustrates a pattern of a portion of an outer frame of a prosthetic valve.

FIG. 15 illustrates a top schematic view of an outer frame shown in FIG. 14 and an inner frame.

FIGS. 16A and 16B illustrate a t-bracket.

FIGS. 17A and 17B illustrate a hinge.

FIGS. 18A and 18B illustrate a ball and socket joint.

FIG. 19 illustrates an overmolding.

FIG. 20 illustrates a top schematic view of an outer frame coupled to an inner frame.

FIG. 21 illustrates a top schematic view of the outer frame shown in FIG. 20.

FIG. 22 illustrates a top schematic view of the inner frame shown in FIG. 20.

FIG. 23 illustrates a side view of the coupling of the outer frame shown in FIG. 21 to the inner frame shown in FIG. 22.

FIG. 24 illustrates a pattern of a valve frame according to an embodiment of the present disclosure.

FIG. 25 illustrates a side cross sectional view of an elongate strut deflected away from an inner frame, for the pattern shown in FIG. 24.

FIG. 26 illustrates the pattern of the valve frame shown in FIG. 24 with the elongate struts deflected away from the inner frame.

FIG. 27 illustrates a top schematic view of the valve frame shown in FIG. 26.

FIG. 28 illustrates a top schematic view of the valve frame shown in FIG. 27 with a skirt positioned on the elongate struts.

FIG. 29 illustrates a cross sectional view of a portion of a sealing body.

FIG. 30 illustrates a pattern of a valve frame according to an embodiment of the present disclosure.

FIG. 31 illustrates an embodiment of a prosthetic valve leaflet.

FIG. 32A illustrates a side view of a micropattern for application to a prosthetic valve leaflet or another portion of a prosthetic valve.

FIG. 32B illustrates a perspective view of the micropattern of FIG. 32A applied to a prosthetic valve leaflet.

FIG. 33A illustrates a side view of a micropattern for application to a prosthetic valve leaflet or another portion of a prosthetic valve.

FIG. 33B illustrates a perspective view of the micropattern of FIG. 33A applied to a prosthetic valve leaflet.

FIG. 34 illustrates a view of a micropattern for application to a prosthetic valve leaflet or another portion of a prosthetic valve.

FIG. 35 illustrates a view of a micropattern for application to a prosthetic valve leaflet or another portion of a prosthetic valve.

FIG. 36 illustrates a view of a micropattern for application to a prosthetic valve leaflet or another portion of a prosthetic valve.

FIG. 37 illustrates a view of a micropattern for application to a prosthetic valve leaflet or another portion of a prosthetic valve.

FIG. 38 illustrates a side cross sectional view of a portion of a sealing skirt including one or more microbeads.

FIG. 39 illustrates an assembly view of an anchor of a prosthetic valve.

FIG. 40 illustrates a cross sectional view of the anchor shown in FIG. 39.

FIG. 41 illustrates a perspective view of a prosthetic valve configured to be deployed to an aortic valve.

FIG. 42 illustrates a top view of the prosthetic valve shown in FIG. 41 with prosthetic leaflets closed.

FIG. 43 illustrates a top view of the prosthetic valve shown in FIG. 41 with prosthetic leaflets opened.

FIG. 44 illustrates a schematic side cross sectional view of the prosthetic valve shown in FIG. 41.

FIG. 45 illustrates a perspective view of a prosthetic valve.

FIG. 46 illustrates a side cross sectional view of a portion of the prosthetic valve along line 46-46 shown in FIG. 45.

FIG. 47 illustrates a view of a plurality of prosthetic valve leaflets flattened.

FIG. 48 illustrates a view of three prosthetic valve leaflets flattened.

FIG. 49 illustrates a view of a single prosthetic valve leaflet flattened.

FIG. 50 illustrates a detail view of end portions of a support ring.

FIG. 51 illustrates a top view of a support ring.

FIG. 52 illustrates a top view of the support ring shown in FIG. 51 with a varied diameter.

FIG. 53 illustrates a side schematic view of a delivery apparatus approaching an implantation site.

FIG. 54 illustrates a side schematic view of a prosthetic implant being deployed to a native valve.

FIG. 55 illustrates a side schematic view of a prosthetic implant deployed to a native valve.

### DETAILED DESCRIPTION

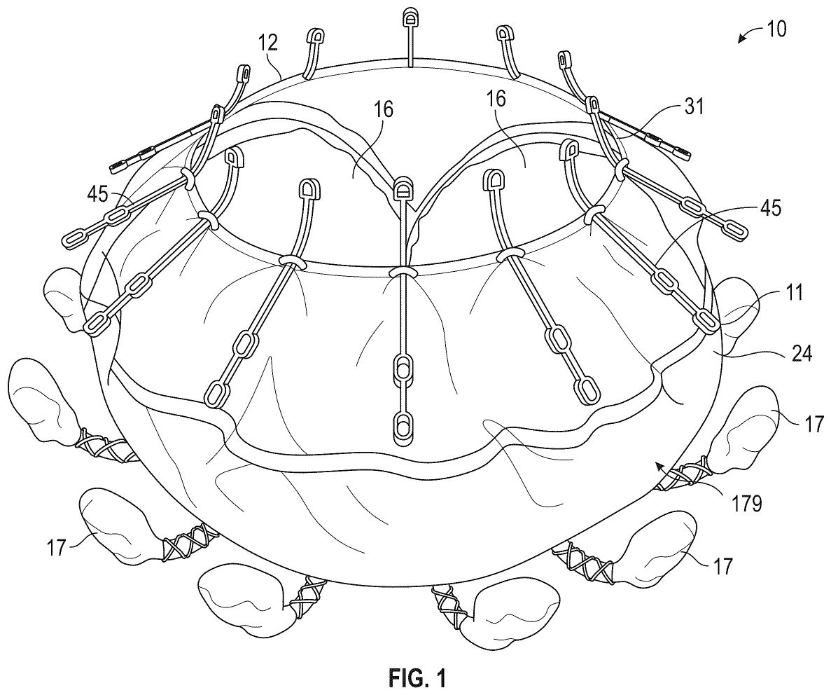

FIG. 1 illustrates a perspective view of a prosthetic valve 10 in the form of a replacement heart valve. The prosthetic valve 10 may be configured to be deployed within a portion of a patient's body. The prosthetic valve 10, for example, may be deployed to an annulus of a native valve, which may comprise a native mitral valve or a native tricuspid valve. In embodiments other implantation locations may be utilized such as within an aortic or pulmonary valve, or in other valves or locations within a patient's body as desired.

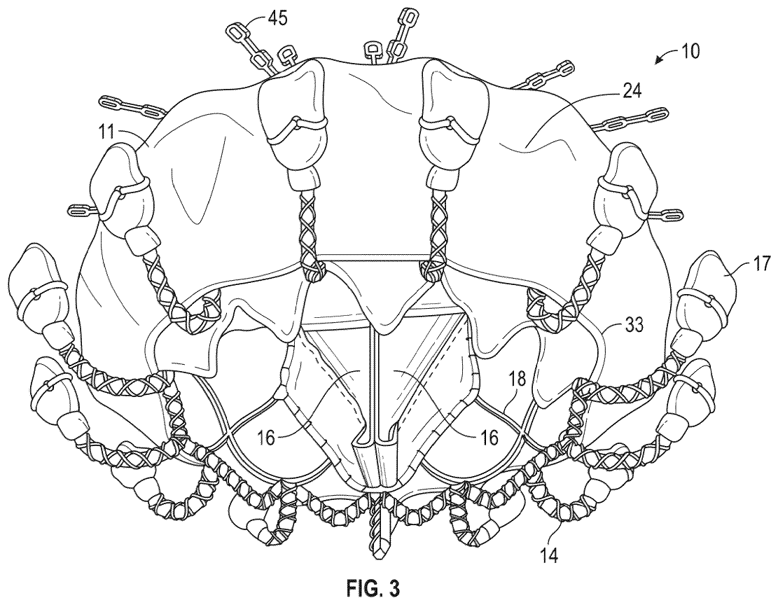

The prosthetic valve 10 may include a proximal end 12 and a distal end 14 (marked in FIG. 3), and a length therebetween. The prosthetic valve 10 may further include one or more prosthetic valve leaflets 16, or a plurality of prosthetic valve leaflets 16, configured to surround a flow channel for controlling flow through the valve 10. The prosthetic valve leaflets 16 may be configured to move between opened and closed states to mimic and replace the operation of native valve leaflets.

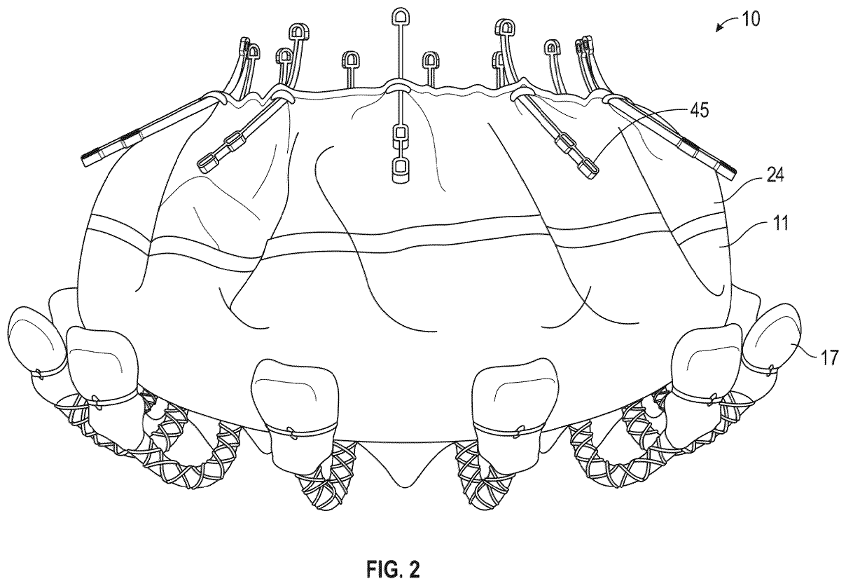

FIG. 2 illustrates a side view of the prosthetic valve 10 and FIG. 3 illustrates a lower perspective view of the prosthetic valve 10.

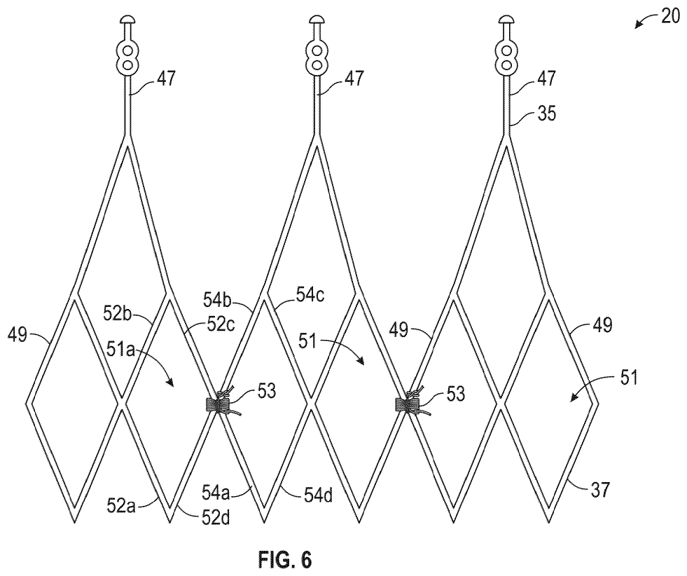

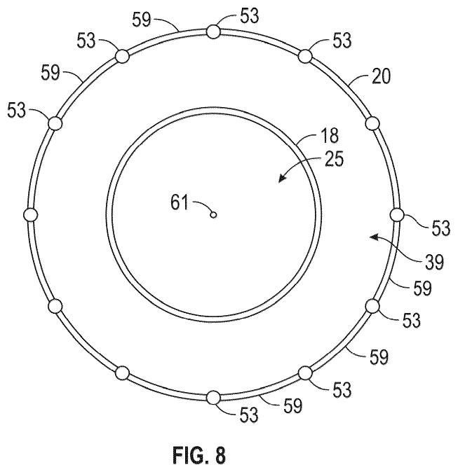

In embodiments, the prosthetic valve leaflets 16 may be coupled to a frame. The frame may include a valve frame or inner frame 18 as shown in FIG. 4 and may include an outer frame 20 as shown in FIG. 6, which may be part of a sealing body 11 and may be spaced from the inner frame 18. FIG. 4 illustrates a side view of the inner frame 18 separated from other components of the prosthetic valve 10. Referring to FIG. 4, the inner frame 18 may include a proximal portion including a proximal end 19 and a distal portion including a distal end 21. The inner frame 18 may have a curved configuration, comprising a curved body that curves radially outward between the proximal end 19 and the distal end 21, or may have another configuration in embodiments as desired. The inner frame 18 may have a circular shape in embodiments, as represented in the top view of FIG. 8 for example.

The inner frame 18 may include a plurality of struts 23 spaced from each other with spaces 15. Such a configuration may allow the inner frame 18 to move between an undeployed, unexpanded, or linearized configuration to a deployed or expanded configuration. For example, the inner frame 18 may expand radially outward to move to the deployed or expanded configuration, with the length of the inner frame 18 decreasing due to the increased diameter of the inner frame 18. Other configurations of inner frames 18 may be utilized as desired.

The prosthetic valve 10 may include one or more anchors 17 that may be coupled to the prosthetic valve leaflets 16 and each may be configured to anchor to a portion of a patient's heart. The anchors 17 may particularly be configured to anchor to the native valve leaflets of the patient's heart. The anchors 17 may extend around the native valve leaflets to anchor to the native valve leaflets. The anchors 17 may comprise distal anchors positioned at the distal end 14 of the valve 10, or in embodiments may be positioned in another position as desired.

Each anchor 17 may be configured as a protruding arm configured to extend distally and then curve in a proximal direction to the tip of the respective one of the anchors 17. Such a configuration may allow the anchor 17 to extend around a native leaflet and around the distal tip of the leaflet, to hook the native leaflet and be positioned radially outward of an outward facing surface of a leaflet of the native valve. The anchor 17 may thus resist a force applied in the atrial or proximal direction to the valve 10 and may anchor the valve 10 within the native valve annulus. Other configurations of anchors 17 may be utilized in embodiments as desired.

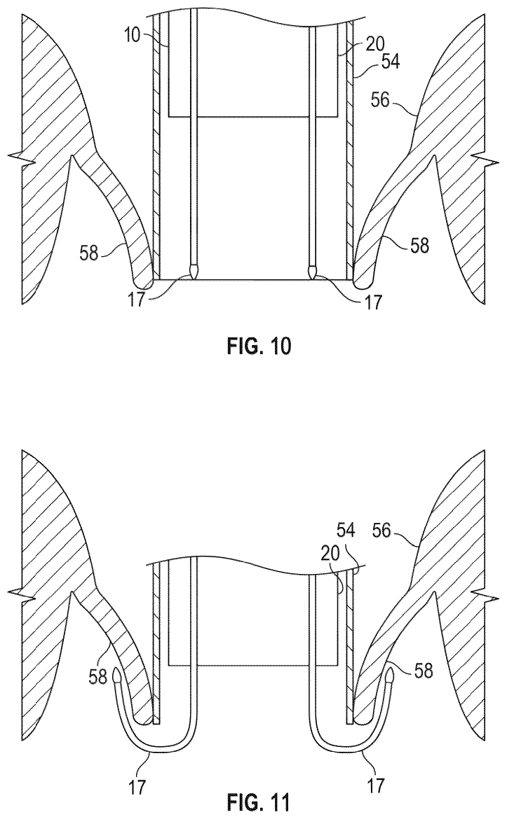

The anchors 17 are shown in FIGS. 1-5 in a deployed or expanded configuration, in which the tips of the anchors 17 extend proximally. In embodiments, the anchors 17 may be configured to be in undeployed, unexpanded, or linearized configuration in which the tips of the anchors 17 extend distally. Such a configuration is shown in FIG. 10 for example. Upon deployment, the anchors 17 may be configured to move from the undeployed configuration radially outward to the deployed configuration, with the tips flipped towards the proximal direction. Such an operation may allow the anchors 17 to flip over the native valve leaflets to anchor to the native valve leaflets during deployment. Such a configuration is shown in FIG. 11 for example. Other deployment methods for the anchors 17 may be utilized in embodiments as desired.

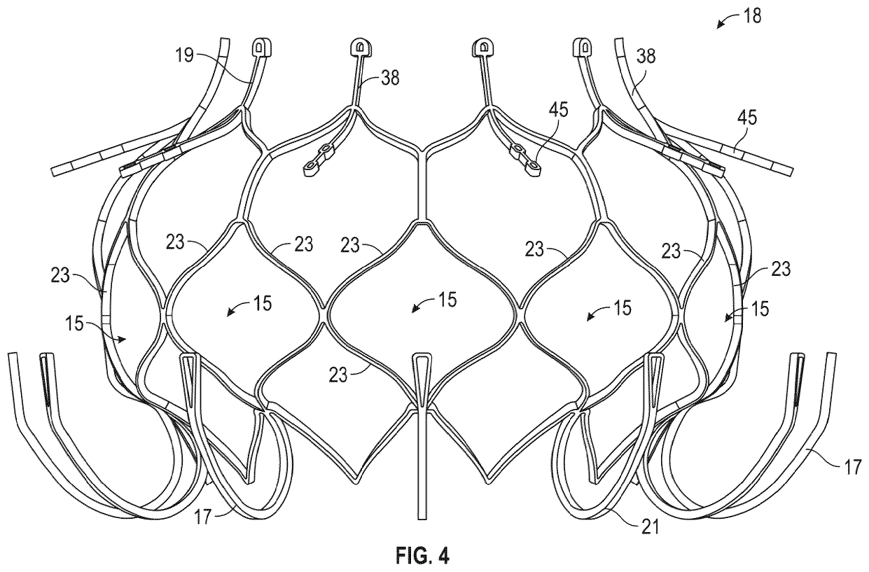

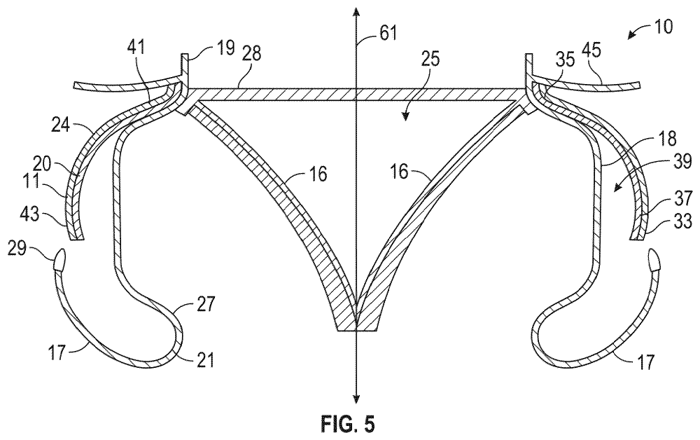

FIG. 5 illustrates a cross sectional view of the prosthetic valve 10. The proximal portion of the inner frame 18 may be coupled to a proximal portion of the plurality of prosthetic valve leaflets 16. The inner frame 18 may support the prosthetic valve leaflets 16. The prosthetic valve leaflets 16 may be coupled to the inner frame 18 and may extend radially inward from the inner frame 18. The prosthetic valve leaflets 16 may couple to the valve frame 18 via an intermediate body 28 that may support the prosthetic valve leaflets 16 and may couple the leaflets 16 to the inner frame 18 via sutures or another method as desired.

The prosthetic valve leaflets 16 may surround a flow channel 25 as marked in FIG. 5 and may move between open and closed states to control flow through the flow channel 25. As shown in FIG. 5, the proximal end of the prosthetic valve 10 may comprise an inflow end of the valve 10, and the distal end of the prosthetic valve 10 may comprise an outflow end, although other configurations may be utilized as desired. The prosthetic valve leaflets 16 may be positioned around a central axis 61 of the prosthetic valve 10. The inner frame 18 and outer frame 20 may each surround the central axis 61 of the prosthetic valve 10.

The anchors 17 may each extend radially outward from the flow channel 25 and radially outward from the prosthetic valve leaflets 16 of the valve 10. FIG. 5, for example, illustrates that the anchors 17 may be coupled to the distal portion of the inner frame 18. The anchors 17 may each include a proximal portion 27 and a distal portion 29, with the proximal portion 27 coupled to the inner frame 18 and the distal portion 29 comprising a tip of the respective anchor 17. The anchors 17 may extend vertically from the proximal portion 27 to the tip at the distal portion 29 when the valve 10 is deployed.

In embodiments, the prosthetic valve 10 may include proximal anchors 45 that may be utilized to secure the prosthetic valve within the native valve. Such anchors 45 are shown in FIGS. 1-5 and may be coupled to a proximal portion of the inner valve frame 18 or may be at another position as desired.

Referring again to FIG. 1, the prosthetic valve 10 may include a sealing body 11. The sealing body 11 may be positioned radially outward from the prosthetic valve leaflets 16 and may comprise the outer surface of the valve 10. The sealing body 11 may define the outer diameter of the valve 10 and may comprise the outer periphery of the valve 10. The sealing body 11 may include a proximal portion having a proximal end 31 and may include a distal portion having a distal end 33 (marked in FIGS. 3 and 5).

Referring to FIG. 5, the sealing body 11 may include a frame 20 (also marked in FIG. 6) and a sealing skirt 24 (also shown in FIGS. 1-3), or in embodiments may comprise only a frame or only a sealing skirt as desired. The frame 20 may comprise an outer frame that is positioned radially outward from the inner frame 18. The sealing skirt 24 may be coupled to the outer frame 20 and may comprise the outer portion of the sealing body 11 as shown in FIG. 1.

The outer frame 20 comprises at least a portion of the sealing body 11 that is configured to apply a seal to a portion of a heart. The outer frame 20 may have a proximal portion 35 that couples to the proximal end 19 of the inner frame 18. The proximal portion 35 may extend radially outward from the proximal end 19 of the inner frame 18 and from the prosthetic valve leaflets 16. A distal portion 37 of the outer frame 20 may be spaced from the prosthetic valve leaflets 16 and the inner frame 18 at a gap 39. The gap 39 may be positioned between the outer frame 20 of the sealing body 11 and a distal portion of the inner frame 18. The inner frame 18 accordingly may comprise an inner frame and the frame 20 of the sealing body 11 may comprise an outer frame positioned radially outward of the inner valve frame 18 and surrounding the inner frame 18 and the prosthetic valve leaflets 16.

As shown in FIG. 5, the outer frame 20 may have a length that extends distally to a lesser distance than the distal end of the inner frame 18. As such, the outer frame 20 may be shorter than the inner frame 18. The outer frame 20 may further have a curved configuration that curves outward from the inner frame 18, with a greatest diameter of the outer frame 20 being at the distal portion of the outer frame 20.

The outer frame 20 of the sealing body 11 may include a plurality of struts 49 (as marked in FIG. 6) forming the frame 20, with spaces 51 between the struts. Such a configuration utilized with the frame 20 may allow the frame 20 to move between an undeployed, unexpanded, or linearized configuration to a deployed or expanded configuration as shown in FIG. 1, in which the outer frame 20 and sealing body 11 have a curved bulbous shape. As with the valve frame 18, the length of the outer frame 20 of the sealing body 11 may decrease as the diameter of the outer frame 20 of the sealing body 11 increases during deployment. The diameter of the outer frame 20 of the sealing body 11 may radially expand outward from the inner valve frame 18 simultaneously, or at a different time or rate of expansion as the inner valve frame 18 in embodiments.

The sealing body 11 may include a sealing skirt 24 (as shown in FIG. 1) that may extend around the inner valve frame 18 and the prosthetic valve leaflets 16. The skirt 24 may be coupled to the frame 20 of the sealing body or may be free from the frame 20 in embodiments.

The sealing skirt 24 may have a proximal portion 41 (marked in FIG. 5) that is coupled to the proximal portion of the frame 20 of the sealing body 11 and may be coupled to the proximal portion of the inner frame 18. The skirt 24 may have a distal portion 43 (marked in FIG. 5) that may be coupled to the distal end of the frame 20, and in embodiments may be coupled to the inner valve frame 18 or one or more of the anchors 17. As shown in FIG. 5, the anchors 17 may be configured to extend radially outward from the inner valve frame 18 and across the gap 39 to the tip of the respective anchor 17.

The sealing skirt 24 may be made of a material that resists fluid flow therethrough, such as a cloth material, woven material, or other material such as a polymer or other material that resists fluid flow therethrough. The material may comprise a fabric. A variety of materials may be utilized for the skirt 24 as desired.

The sealing body 11 may be configured to abut a portion of the patient's heart to reduce fluid flow. The skirt 24 may be configured to seal a portion of the native valve annulus. For example, the sealing body 11 may abut a surface of a patient's native valve leaflet to reduce fluid flow between the sealing body 11 and the native leaflet. The sealing body 11 may be configured to abut other portions of the patient's heart to reduce fluid flow as desired.

FIG. 6 illustrates a view of a construction of the outer frame 20. The construction is shown as a flat pattern, however the pattern shown in FIG. 6 would be wrapped around the inner frame 18 to form a bulbous shape in embodiments. Further, only a portion of the outer frame 20 is shown, with the pattern and use of rotation joints 53 being repeated to a desired amount.

The proximal portion 35 of the outer frame 20 may include struts 47 configured to couple to corresponding struts 38 of the inner frame 18 as marked in FIG. 4. Both struts, for example, may include eyelets that allow the outer frame 20 to couple to the inner frame 18. The outer frame 20 accordingly may couple to the proximal end 19 of the inner frame 18 to maintain an outward biasing force by the outer frame 20. The distal end of the outer frame 20 may remain uncoupled to the inner frame 18.

The struts 49 of the outer frame 20 may form one or more strut cells that surround the spaces 51 between the struts 49. For example, struts 52a—d form a strut cell surrounding the interior space 51a. The cells may be positioned adjacent to each other as shown in FIG. 6.

The cells may form columns of the outer frame 20 that extend from the proximal portion of the outer frame 20 to the distal portion 37 of the outer frame 20. The columns, for example, may comprise the columns of strut cells that are adjacent to each other as shown in FIG. 6.

Adjacent strut cells or struts 49, for example, may comprise adjacent portions of the outer frame 20. The portions may each include at least one strut cell in embodiments.

In embodiments, portions of the outer frame 20 may be coupled to each other with one or more rotation joints 53 as shown in FIG. 6. The rotation joints 53 may couple the portions to each other and allow the portions to rotate relative to each other. FIG. 6, for example, illustrates the rotation joints 53 as sutures that couple adjacent portions of the outer frame 20 to each other. The rotation joints 53 may couple together adjacent strut cells, such as the strut cell formed by struts 52a—d that is coupled to and adjacent to the strut cell formed by struts 54a—d. The strut cell formed by struts 52a— d, for example, may comprise a first portion of the outer frame 20, and the strut cell formed by struts 54a—d, for example, may comprise a second portion of the outer frame 20. The strut cell formed by struts 52a—d and the strut cell formed by struts 54a—d each comprise adjacent columns of the outer frame 20. In embodiments, other portions of the outer frame 20 may be coupled to each other with one or more rotation joints 53.

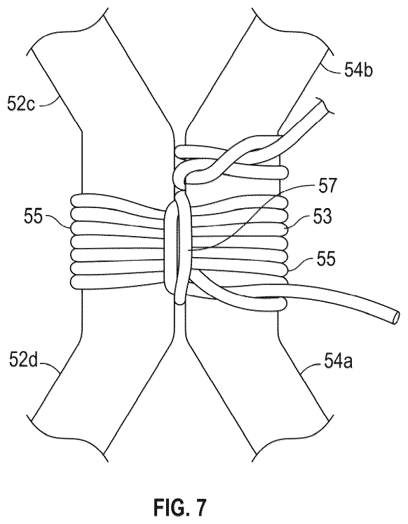

FIG. 7 illustrates a close up view of one of the rotation joints 53 coupling adjacent struts 52c, 52d, 54a, and 54b to each other. The struts 52c, 52d may be non-integral with the struts 54a, 54b, yet may be coupled together via sutures forming a suture joint between the adjacent struts. The adjacent strut cells formed by struts 52a—d and formed by struts 54a—d may accordingly be coupled to each other and configured to rotate relative to each other. The sutures may be made of a stiff material or elastic material in embodiments.

The suture joint may comprise a knot, which may be a variety of forms of knots. A knot may comprise a shear-lashing knot as shown in FIG. 7. The shear-lashing knot, for example, may include horizontal wraps 55 of the suture material and one or more vertical wraps 57 that wrap over the horizontal wraps and between the adjacent struts 52c, 52d and 54a, 54b. The horizontal wraps 55 may enable the adjacent portions to rotate, and the vertical wraps 57 may allow for balanced compression. As such, the shear lashing knot may allow the struts to rotate relative to each other, yet reduce the possibility of slippage between the adjacent struts. The number of wraps may be varied depending on the degree of rotation desired and the controlled compression required. Other forms of shear-lashing knots, or other knots may be utilized in embodiments as desired.

The rotation joints 53 may couple portions of the outer frame 20 to each other at a variety of circumferentially spaced portions of the outer frame 20. FIG. 8, for example, illustrates a top schematic view of the outer frame 20 extending around the inner frame 18 and spaced from the inner frame 18 with the gap 39 shown in FIG. 5. Other features of the prosthetic valve 10 are excluded from view in FIG. 8. The rotation joints 53 are represented as nodes that are positioned between adjacent portions 59 of the outer frame 20 and allow the adjacent portions 59 to rotate relative to each other. The portions 59 may comprise the strut cells or struts shown in FIG. 6, for example, or another portion of the outer frame 20. In embodiments, a sealing skirt 24 may be coupled to the outer frame 20 to seal with a portion of a native heart annulus, as shown in FIG. 1 for example.

The rotation joints 53 may be equally spaced from each other, or a varied spacing may be provided as desired. Twelve rotation joints 53 are shown in FIG. 8, although in embodiments a greater or lesser number may be utilized as desired. The rotation joints 53 may be configured to allow the portions 59 to rotate in a plane extending transverse to the central axis 61 of the prosthetic valve 10 that the inner frame 18 and outer frame 20 surround. As such, the portions 59 may rotate radially inward or outward relative to the central axis 61 and relative to the inner frame 18. In embodiments, however, the rotation joints 53 may be configured to allow for rotation towards or away from a proximal or distal portion of the outer frame 20.

The rotation joints 53 may allow the portions 59 of the outer frame 20 to rotate, to improve conformability of the outer frame 20 to a shape of a native valve annulus. The outer frame 20 may be configured to conform to a shape of an annulus of the native valve. The native valve annulus, for example, may not have a shape of a perfect circle, and may have other shapes such as other circular shapes and ovoid shapes such as oval shapes or elliptical shapes, which may include recesses. Points of calcification may further vary the shape of the native valve annulus. The rotation joints 53 may improve the ability of the portions 59 to rotate to conform to the native shape, rather than presenting a rigid circle shape to the native annulus.

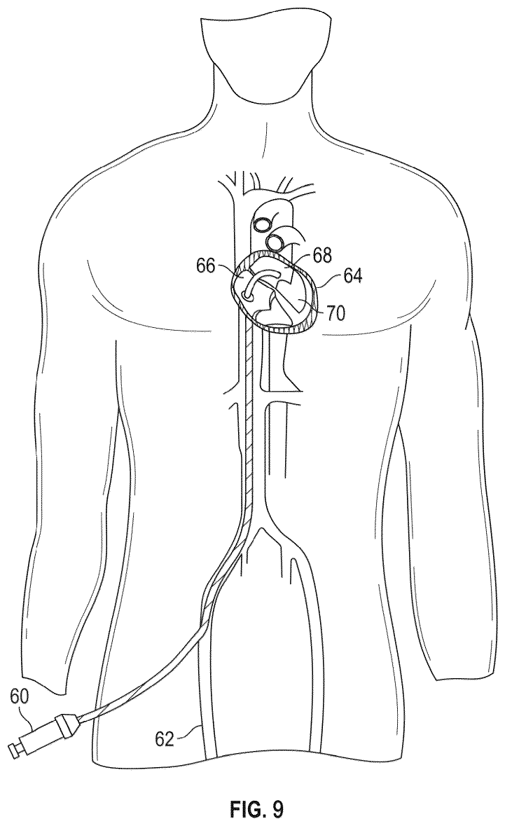

FIG. 9 for example, illustrate an exemplary method of deploying the prosthetic valve 10. Referring to FIG. 9, a delivery apparatus 60 may be passed percutaneously into a patient's body in a minimally invasive manner. In other embodiments, more invasive means may be utilized as desired.

The delivery apparatus 60 may be utilized for transcatheter delivery of the valve. The delivery apparatus 60 may pass transvenous through the femoral artery 62 or another portion of the patient's vasculature. For example, transjugular entry or other methods of entry may be utilized as desired. The delivery apparatus 60 may pass to the patient's heart 64.

The delivery apparatus 60 may be used to deliver the valve to the tricuspid valve, and as such, may be positioned within the right atrium 66 of the patient's heart for delivery to the tricuspid valve. In an embodiment in which delivery is to the mitral valve, the delivery apparatus 60 may pass transseptal to the left atrium 68 for delivery to the mitral valve. The delivery apparatus 60 may advance towards the left ventricle 70 of the patient's heart for mitral delivery.

The prosthetic valve 10 may then be deployed, with a capsule of the delivery apparatus 60 being retracted relative to the prosthetic valve 10. FIG. 10, for example, illustrates the prosthetic valve 10 positioned within a capsule 54 of the delivery apparatus 60 and in position to deploy the prosthetic valve 10 to the native valve within the native valve annulus 56. The anchors 17 may be in position to extend around the distal tips of the leaflets 58 to anchor the prosthetic valve 10 within the native valve annulus 56.

FIG. 11 illustrates the capsule 54 being retracted relative to the prosthetic valve 10 to allow the anchors 17 to extend radially outward from the capsule 54 and extend around the distal tips of the leaflets of the native heart valve.

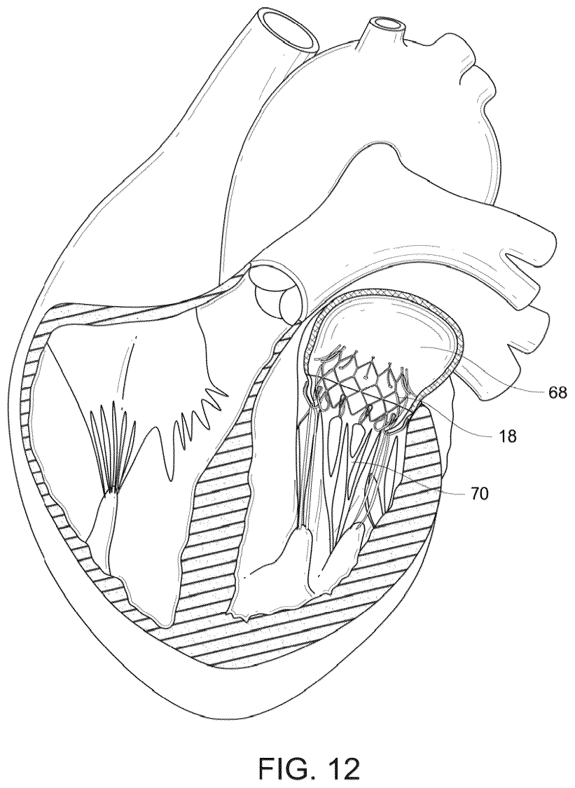

FIG. 12 illustrates the valve frame 18 deployed to the native valve annulus, with other portions of the prosthetic valve 10 excluded from view for clarity.

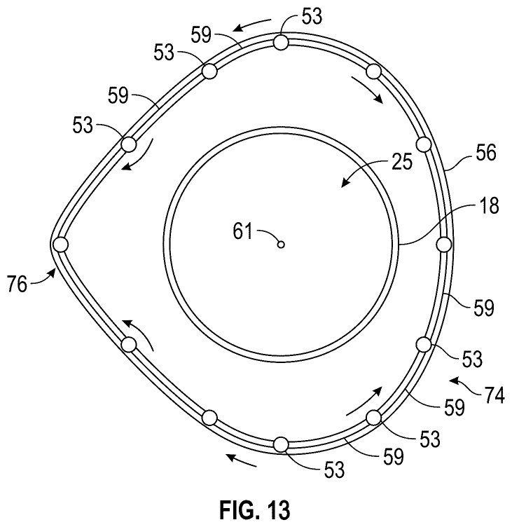

The native valve annulus 56, such as the native mitral valve annulus, may not have the shape of a perfect circle. The shape may include ovoid or other shapes, which may include recesses. FIG. 13, for example, illustrates a top schematic view of the prosthetic valve 10 deployed to a native valve annulus 56 that does not have the shape of a perfect circle. A portion 74 of the native valve annulus 56 may have an ovoid shape, and a portion 76 may include a recess.

Thus, upon deployment, the portions 59 of the outer frame 20 may be allowed to rotate relative to each other in response to a shape of an annulus of the native valve. The frame 20 may be allowed to conform to the shape of the annulus of the native valve.

The rotation joints 53 may allow the portions 59 of the outer frame 20 to rotate relative to each other, to conform (whether fully or partially) to the shape of the native valve annulus 56. The portions 59 may rotate relative to each other to vary a shape of an outer surface of the frame 20 and the prosthetic valve 10. Each portion 59 may either deflect radially inward or outward relative to the inner frame 18. Various portions of the outer frame 20 may rotate either inward or outward, with one portion rotating inward and another portion possibly rotating outward. The inner frame 18 may retain its shape during the rotation of the portions 59 of the outer frame 20. For example, the inner frame 18 may retain a circular shape. The rotation of the portions 59 of the outer frame 20 may further allow a sealing skirt 24 (marked in FIG. 1) to have a varied shape that conforms (whether fully or partially) to the shape of the native valve annulus 56. The seal of the prosthetic valve 10 to the native valve annulus 56 accordingly may be improved.

The rotation may continue during the cardiac cycle, thus allowing the outer frame 20 to more accurately following the anatomical geometry of the annulus through the cardiac cycle.

The proximal portion of the outer frame 20 may remain coupled to the proximal end of the valve frame 18 to maintain an outward biasing force by the outer frame 20 against the native valve annulus.

Various other configurations of the outer frame 20 and the rotation joints may be utilized. FIG. 14, for example, illustrates an embodiment in which each juncture point of the struts 78 includes a rotation joint 53. As such, increased rotation and flexibility of the outer frame 80 may result. FIG. for example, illustrates a top schematic view showing the increased number of rotation joints 53 that couple portions 82 of the outer frame 80 to each other.

FIGS. 16A and 16B illustrate an embodiment of a rotation joint comprising a t-bracket 84. The t-bracket 84 may couple adjacent portions 86 of the outer frame and allow the portions 86 to rotate relative to each other. A central support 88 of the t-bracket 84 may reduce the possibility of slippage between the portions 86.

A rotation joint such as a t-bracket may support the frame 20 during procedural loading into the delivery apparatus of a delivery system, during deployment, and during recapture, as necessary. The t-bracket may reduce the degrees of freedom to prevent rotation of the rotation joints inward. Such a single-direction of rotation may ensure that the outer frame 20 behaves as desired, producing a radial outward force for annular sealing, local flexibility and reduced slippage between struts during loading into and deployment from the delivery system. Any of the embodiments of rotation joints as disclosed herein may include a single direction of rotation, or single degree of freedom as desired.

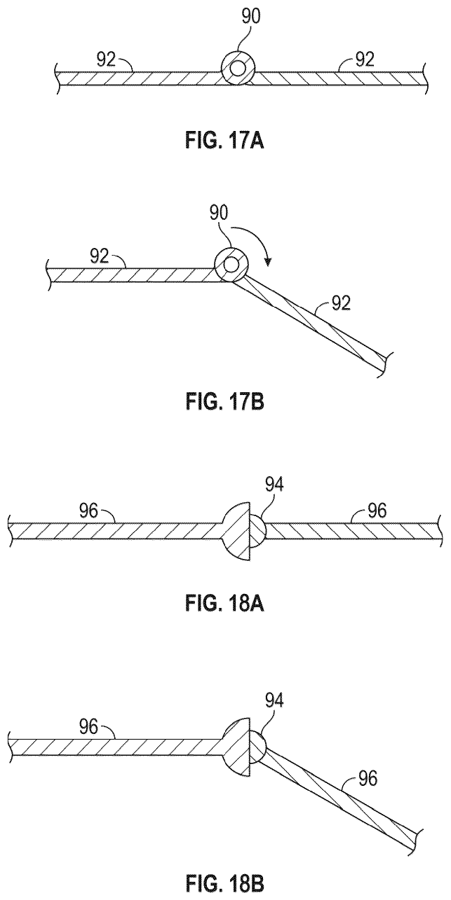

FIGS. 17A and 17B illustrate an embodiment of a rotation joint comprising a hinge 90. The hinge 90 may couple adjacent portions 92 of the outer frame and allow the portions 92 to rotate relative to each other. A hinge 90 may be formed that only allows for motion in a single plane.

FIGS. 18A and 18B illustrate an embodiment of a rotation joint comprising a ball and socket joint 94. The ball and socket joint 94 may couple adjacent portions 96 of the outer frame and allow the portions 96 to rotate relative to each other. Multiple degrees of freedom may be provided with a ball and socket joint 94, including rotation radially inward and outward, and deflection towards or away from a proximal or distal end of the prosthetic valve.

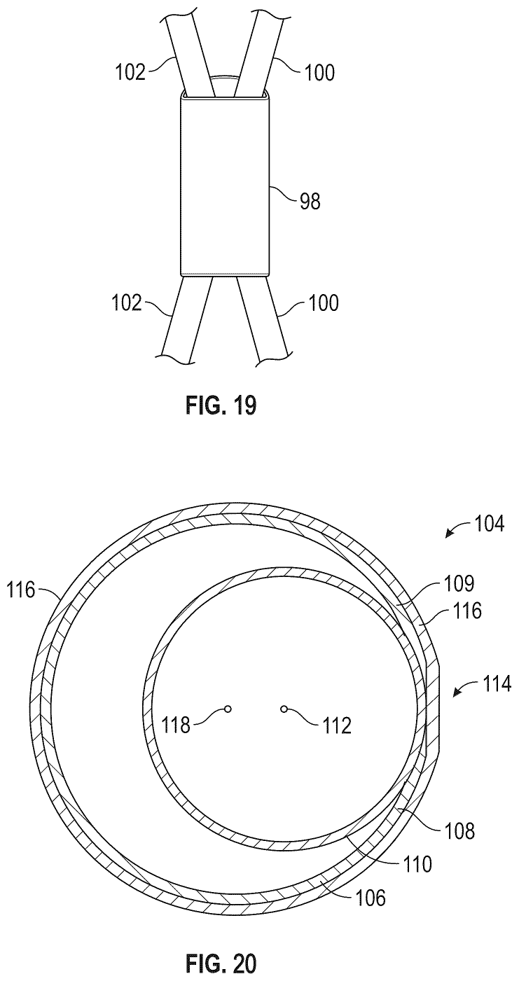

FIG. 19 illustrates an embodiment of a rotation joint comprising an overmolding 98. The overmolding 98 may comprise a tube of material (such as shrink-tubing or other material) that may be positioned to wrap around and couple adjacent portions 100, 102 of the outer frame, and allow the portions 100, 102 to rotate relative to each other. The overmolding 98 may comprise a plastic and/or silicone material or another form of material. The portions 100, 102 may comprise struts of the outer frame, as shown in FIG. 19.

Combinations of types of rotation joints may be utilized in embodiments, or a single type of rotation joint may be utilized as desired. As such, in embodiments, a rotation joint may include one or more of a suture joint, a t-bracket, a ball and socket joint, an overmolding, or a hinge, or other forms of rotation joints. Any of the embodiments of rotation joints as disclosed herein may include a single direction of rotation, or single degree of freedom as desired. In embodiments, other articulation points may be utilized to increase the degrees of freedom as desired.

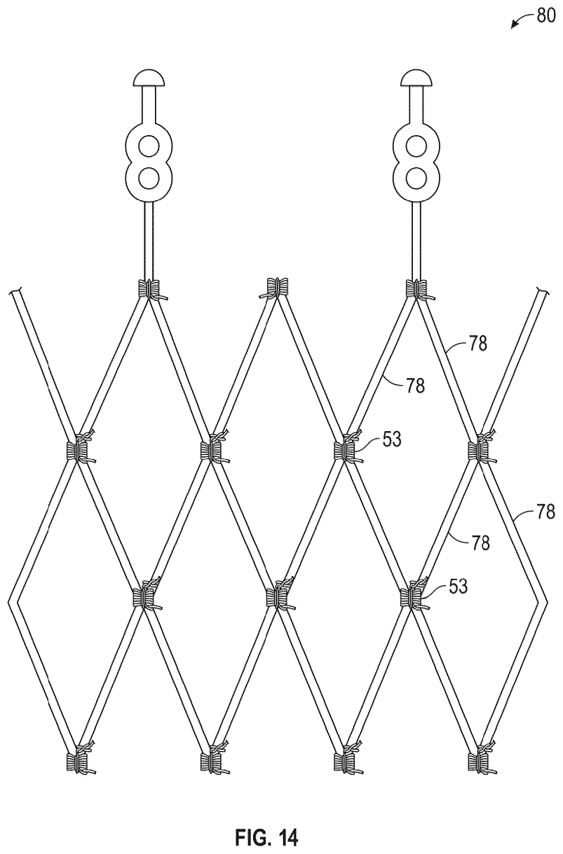

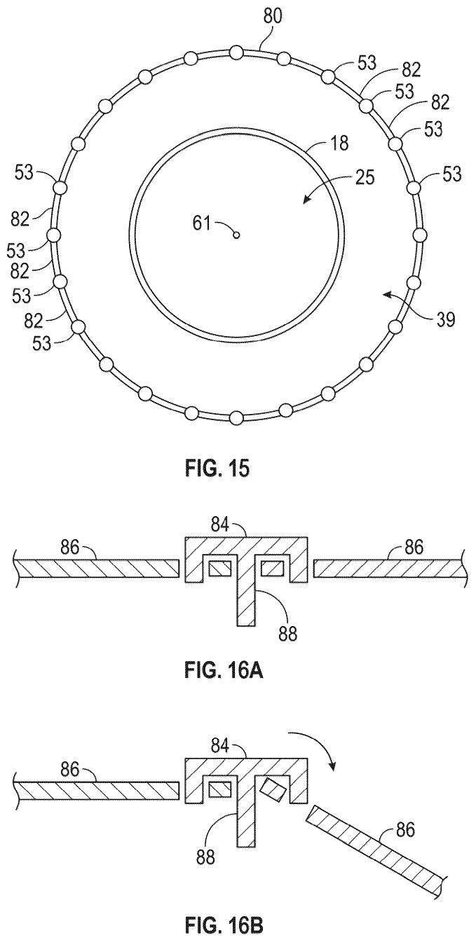

The embodiments of FIGS. 1-19 may beneficially allow for improved conformability of the embodiments of prosthetic valves to the native valve, among other benefits. Reduced possibility of paravalvular leakage may result. Various modifications of the embodiments may be provided, including substitution or addition of features across various embodiments disclosed herein. The embodiments of FIGS. 1-19 may be utilized solely or in combination with features of other embodiments disclosed herein.

FIG. 20 illustrates a top schematic view of an embodiment of a prosthetic valve 104 including an outer frame 106 having a crescent shape with two ends 108, 109 circumferentially spaced from each other. The prosthetic valve 104 may include an inner frame 110 supporting one or more, or a plurality of prosthetic valve leaflets, and surrounded by at least a portion of the outer frame 106 and coupled to each of the two ends 108, 109 of the outer frame 106.

The inner frame 110 may have a circular shape, as shown in FIG. 20 (and in FIG. 22, in which the inner frame 110 is shown separate from the outer frame 106). The inner frame 110 may surround a center 112 of the inner frame 110, which may be positioned within of a flow channel of the inner frame 110. The inner frame 110 may be configured similarly as the inner frame 18 shown and discussed with respect to FIGS. 1-5, and may be coupled to the prosthetic valve leaflets, and may include distal anchors (as shown in FIG. 1) for example, for anchoring the prosthetic valve 104 to the native valve. The distal anchors, for example, may be configured to extend around one or more leaflets of a native valve to anchor the prosthetic valve to the native valve. Features of the prosthetic valve 104 are excluded from view for clarity in FIG. 20.

The outer frame 106 may have a larger diameter than the diameter of the inner frame 110 and may have a crescent shape such as a “C” shape or other form of crescent shape, and may form an outer frame periphery of the prosthetic valve 104. The outer frame 106 may comprise a portion of a sealing body that extends around a portion of the inner frame 110, similar to the outer frame 20 shown in FIGS. 1-5. The outer frame 106, however, may extend around a portion of the inner frame 110. The ends 108, 109 of the outer frame 106 may couple to the inner frame 110 such that a portion 114 of the inner frame 110 between the two ends 108, 109 is uncovered by the outer frame 106 and thus the portion 114 forms the outermost frame surface of the prosthetic valve 104 at that position. A sealing skirt 116 may be coupled to and extend around the outer frame 106 and the portion 114 of the inner frame 110 between the ends 108, 109 to form an outer surface of the prosthetic valve 104, similar to the sealing skirt 24 shown in FIGS. 1-5.

As shown in FIG. 20, a center 118 of the outer frame 106 may be offset from the center 112 of the inner frame 110. The frames 106, 110 may be non-concentric with one another.

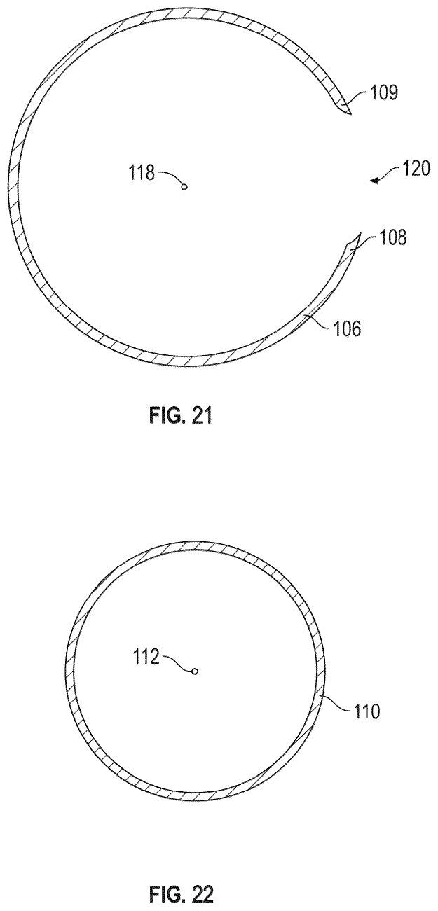

FIG. 21 illustrates a top schematic view of the outer frame 106 separate from the inner frame 110. The space 120 between the ends 108, 109 of the outer frame 106 is shown. The outer frame 106 may have a circular shape that may comprise a portion of a circle as shown in FIG. 21, or other shapes such as ovoid. The shape of the outer frame 106 may be configured such that when the outer frame 106 is joined with the inner frame 110, a “D” shape together may be formed by the outer frame 106 and inner frame 110 due to the varied radii of the outer frame 106 and inner frame 110. FIG. 22, for example, illustrates the inner frame 110 separate from the outer frame 106.

A “D” shape, or other shape that does not comprise a circle, formed by the combination of the outer frame 106 and inner frame 110 may improve positioning of the prosthetic valve 104 within the native valve annulus. For example, the native valve annulus itself may have a “D” shape, and the resulting outer surface shape of the prosthetic valve 104 may have a similar “D” shape that may conform to the shape of the native valve annulus. The “D” shape may conform better to the “D” shape of the native valve annulus better than a circle shape of the prosthetic valve 104. Further, upon deployment of the prosthetic valve 104 to a native valve, the outer frame 106 may have an outer surface that may be configured to move in response to a shape of the annulus to conform to a shape of an annulus of the native valve.

In a configuration in which a “D” shape results, the flattened portion (corresponding to the portion 114 of the inner frame 110) may be positioned at the respective flattened portion of the native valve annulus having a “D” shape. Such a portion may comprise an anterior portion of the native valve annulus. The curved portion of the “D” shape may be positioned at the posterior portion of the native valve annulus. Other configurations may be utilized based on the configuration of the native valve annulus.

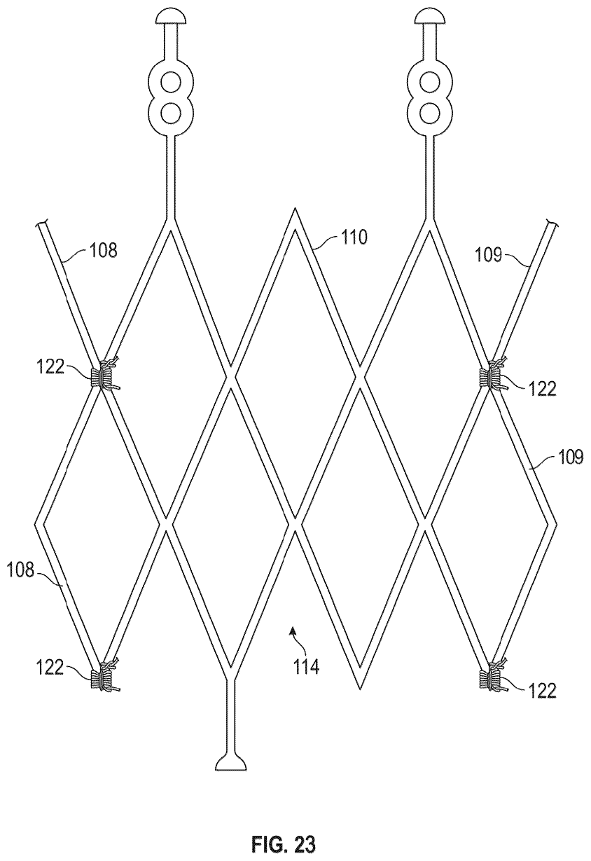

The ends 108,109 of the outer frame 106 may couple to the inner frame 110 in a variety of manners, for example via connectors between the outer frame 106 and inner frame 110. In embodiments, the connectors may comprise other forms of connectors disclosed herein, including the use of rotation joints. FIG. 23, for example, illustrates a side view of a portion of the prosthetic valve showing the ends 108,109 of the outer frame 106 coupling to the inner frame 110 via connectors 122. The connectors 122 may comprise sutures, and may comprise knots, such as a shear-lashing knot in embodiments. Other forms of connectors may be utilized in embodiments.

In embodiments, proximal portions of the outer frame 106 and inner frame 110 may be coupled to each other in a similar manner as the proximal portions of the outer frame 20 and inner frame 18 discussed with respect to FIGS. 1-6.

The proportion of the inner frame 110 that is not covered by the outer frame 106 may be varied in embodiments. For example, in embodiments, at least 10% of the outer circumference of the inner frame may be not covered by the outer frame 106, as well as at least 20%, or at least 30% or at least 40% or another proportion as desired. In embodiments, less than 10% of the outer circumference of the inner frame may be not covered by the outer frame 106, as well as less than 20%, or less than 30% or less than 40% or another proportion as desired. The proportion may be set based on the desired shape of the prosthetic valve to be produced.

In embodiments, both the outer frame 106 and the inner frame 110 may be independently varied in size to modify the resulting shape of the prosthetic valve. For example, the shape and size of a resulting “D” shape may be varied as desired. If a rounded posterior portion of the native valve is large, then a larger outer frame 106 may be utilized. If a flattened anterior portion of the native valve is large, then a larger inner frame 110 may be utilized. Other shapes may be utilized based on the shape of the annulus.

In embodiments, other configurations of outer and inner frames may be utilized in combination. For example, in an embodiment the outer frame may entirely encircle the inner frame 110 and thus may comprise a closed body, having an “0” shape or another form of circular or ovoid shape, rather than the “C” shape shown in FIG. 21. The inner frame 110, however, may be connected to a portion of the interior surface of the outer frame in an offset manner as shown in FIG. 20 for example. The inner frame 110 thus may have a smaller diameter than such an outer frame and may be positioned offset within the outer frame. The center 112 of the inner frame 110 may be offset from the center of the outer frame. In such a configuration, the outer surface of the outer frame may comprise the outermost frame surface of such a valve and may be covered with a sealing skirt for forming a seal with the native valve annulus.

The embodiments of FIGS. 20-23 may beneficially allow for improved conformability of the embodiments of prosthetic valves to the native valve, among other benefits. Reduced possibility of paravalvular leakage and outflow tract obstruction may result. Various modifications of the embodiments may be provided, including substitution or addition of features across various embodiments disclosed herein. The embodiments of FIGS. 20-23 may be utilized solely or in combination with features of other embodiments disclosed herein.

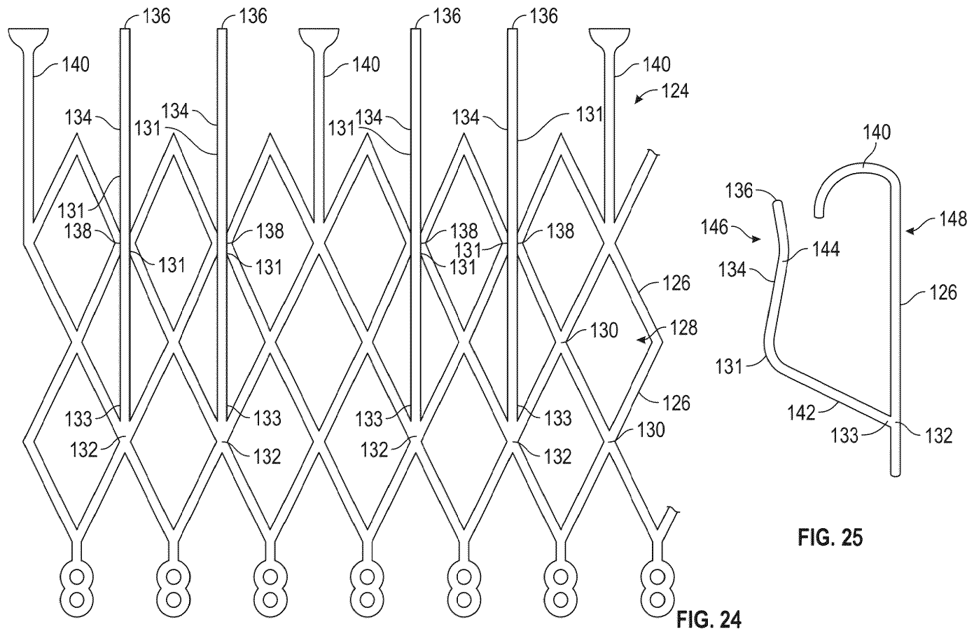

FIG. 24 illustrates a pattern of a frame 124 that may be utilized with a prosthetic valve. The frame 124 may include a plurality of struts 126 that are joined together with spaces 128 between the struts 126. The struts 126 may join at junctures 130 that exist between the struts 126.

The pattern of struts 126, spaces 128, and junctures 130 may result from the formation of the frame 124. The frame 124 may be formed from a unitary piece of material and may be cut from such material to form the configuration of the struts 126, spaces 128, and junctures 130. The unitary piece of material may have a cylindrical shape in embodiments, to result in a cylindrical frame 124 following cutting (although the frame 124 is shown as a flat pattern in FIG. 24).

Elongate struts 134 may be produced that extend distally from junctures 132. During the formation process, the junctures 132 may exist between the struts 126 and the elongate struts 134 may extend distally from such junctures 132. The proximal portions 133 of the elongate struts 134 accordingly may be formed integral with the junctures 132. The elongate struts 134 may extend longitudinally in a distal direction to the distal ends 136 of the elongate struts 134. Cuts 138 may exist between the distal portions 131 of the elongate struts 134 and the adjacent struts 126 such that the distal portions 131 of the elongate struts 134 are cut away at these points. The distal portions 131 accordingly may not be integral with the adjacent struts 126 and may be pulled and deflected away from the adjacent struts 126 at the cuts 138. The proximal portions 133 of the elongate struts 134 accordingly may be integral with the junctures 132, and the distal portions 131 of the elongate struts 134 may be cut-away and non-integral with the remainder of the frame 124.

With the distal portions 131 of the elongate struts 134 being deflectable away from the remainder of the frame 124, in a formation step the distal portions 131 may be deflected away from the remainder of the frame 124. FIG. 25 for example, illustrates the distal portions 131 of the elongate struts 134 being deflected outward from the remainder of the frame 124, to form an outward extending portion 142 and a vertical extending portion 144 that terminates in the distal end 136 of the elongate strut 134. The proximal portion 133 of the elongate strut remains integral with the juncture 132.

With the elongate struts 134 in such a configuration, the elongate struts 134 may form an outer frame 146 extending around the remainder of the frame 124 that may comprise an inner frame 148. The inner frame 148, as such, may be coupled to one or more, or a plurality of, prosthetic valve leaflets and may support such leaflets in a similar manner as the inner frame 18 discussed with respect to FIGS. 1-5.

A method of forming at least a portion of the prosthetic valve may include deflecting the distal portions 131 of each of a plurality of elongate struts 134 of a sealing body away from an inner frame 148 for supporting one or more prosthetic valve leaflets or a plurality of prosthetic valve leaflets. A proximal portion 133 of each of the plurality of elongate struts 134 may be formed integral with the inner frame 148. The inner frame 148 and the plurality of elongate struts 134 may be formed from a unitary piece of material.

FIG. 26 illustrates the inner frame 148 with the elongate struts 134 lifted out of view. A gap may exist where the distal portions 131 of the elongate struts 134 have been lifted out from the inner frame 148. Such a gap may be formed between the adjacent struts of the inner frame 148. Such a gap may be closed with a connector 150, such as a suture connector as disclosed herein, which may be configured as a knot such as a shear-lashing knot. In embodiments, other forms of connectors may be utilized at the junctures as disclosed herein, including one or more of a suture joint, a t-bracket, a ball and socket joint, an overmolding, or a hinge, or other forms of connectors. Rotation joints as may be disclosed herein may be utilized to allow a first portion of an inner frame 148 rotate relative to a second portion of the inner frame 148 in a manner as discussed with respect to the embodiments of FIGS. 1-19. In embodiments, the inner frame 148 may be biased to fill the gap remaining after the distal portions 131 of the elongate struts 134 are deflected from the inner frame 148.

Distal anchors 140 may be shape-set to curve proximally as shown in FIG. 25. The distal anchors 140 may extend from the distal ends of the inner frame 148 and may be configured to extend around one or more leaflets of the native valve to anchor the prosthetic valve to the native valve.

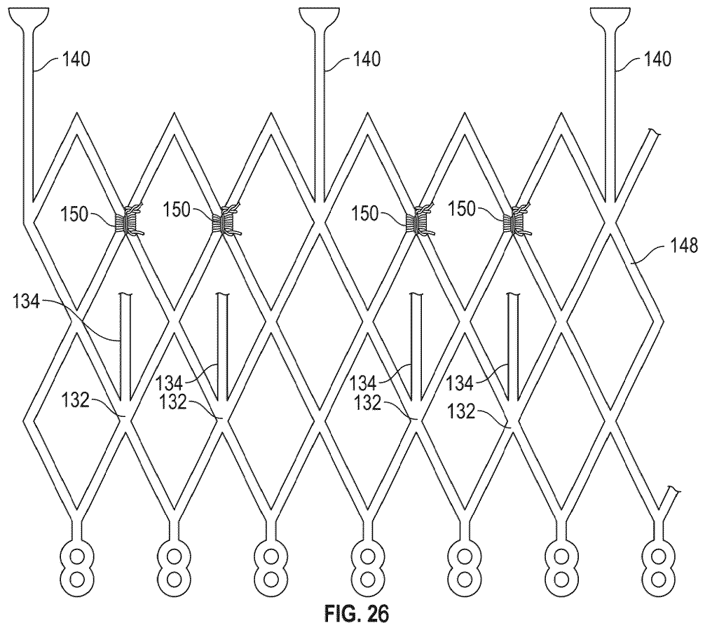

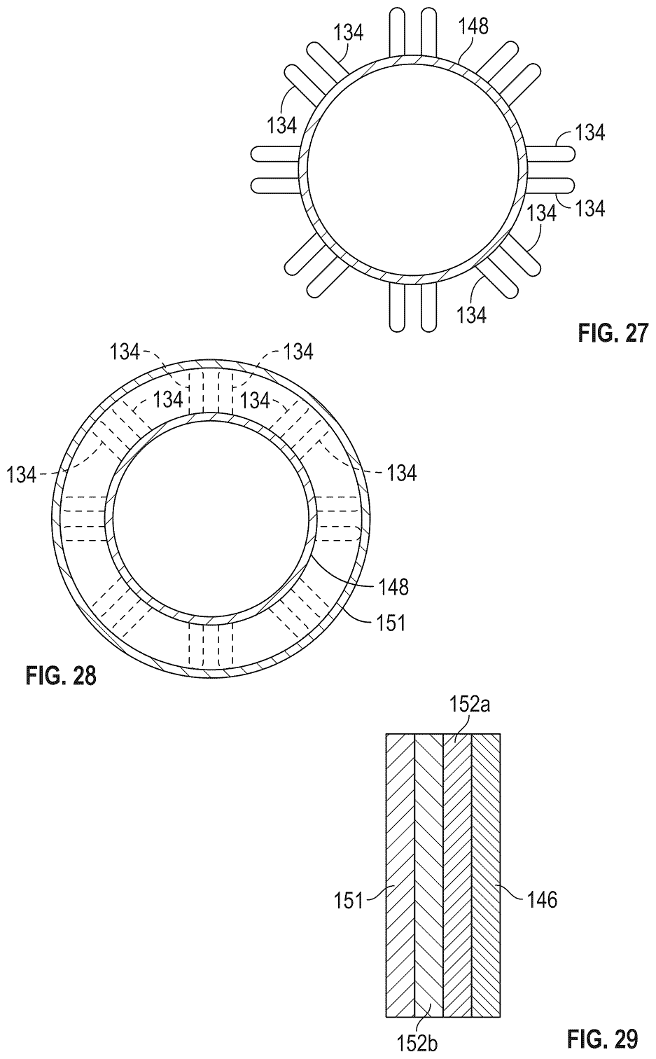

The inner frame 148 and outer frame 146 may be formed into the round bulbous shape shown in FIG. 1 for example. The elongate struts 134 may extend radially outward from the inner frame 148, as represented in the top schematic view of FIG. 27. The elongate struts 134 may be positioned equally from each other, or at varied spacings as desired. In embodiments, a varied number of elongate struts 134 may be utilized. FIG. 28 illustrates that a sealing skirt 151 may be provided that is positioned over the plurality of elongate struts 134 when the elongate struts 134 are deflected away from the inner frame 148. The sealing skirt 151 may form a sealing body that surrounds at least a portion of the inner frame 148 and is configured to seal a portion of an annulus of the native valve. The sealing body may contact a portion of an annulus of a native valve.

The sealing body may include the skirt 151 and the outer frame 146 having the plurality of elongate struts 134. Each of the plurality of elongate struts 134 may have a proximal portion 133 that is integral with the inner frame 148 and a distal portion 131 that is spaced from the inner frame 148. One or more prosthetic valve leaflets may be coupled to the inner frame 148.

The resulting prosthetic valve may have an appearance similar to the prosthetic valve 10 shown in FIG. 1, yet the construction of the prosthetic valve may include the elongate struts 134 integral with the inner frame 148. The outer frame 146 may encircle the inner frame 148 and be utilized to seal with a native valve annulus. The resulting prosthetic valve may be deployed in a similar manner as the prosthetic valve 10 shown in FIG. 1.

Referring to FIG. 29, a cross sectional view of a portion of the sealing body is shown. In embodiments, a compressible material 152a, b, may be positioned between the outer frame 146 and the sealing skirt 151. The compressible material 152a, b, may comprise one more layers of compressible material or other forms of compressible material as desired. FIG. 29 illustrates use of layers of a compressible material, with the sealing skirt 151 forming the outermost surface of the prosthetic valve and the outer frame 146 (comprising the elongate struts 134) being positioned inward of the compressible material 152a, b.

In embodiments, the compressible material 152a, b may comprise foam or another form of compressible material 152a, b. The compressible material 152a, b may serve to compress when a force is applied to the sealing skirt 151 by a native valve annulus, to enhance sealing against the native valve annulus.

FIG. 30 illustrates an alternative configuration of the frame shown in FIG. 24, in which the elongate struts 154 are equally spaced and alternate in position with the distal anchors 156.

The embodiments of FIGS. 24-30 may beneficially provide for improved construction of a prosthetic valve, with an inner and outer frame formed from a single frame pattern. Such a configuration may reduce the complexity of manufacturing a prosthetic valve having an inner frame mechanically joined to an outer frame. Various modifications of the embodiments may be provided, including substitution or addition of features across various embodiments disclosed herein. The embodiments of FIGS. 24-30 may be utilized solely or in combination with features of other embodiments disclosed herein.

In embodiments herein, a surface roughness of at least a portion of a prosthetic valve may be altered. The prosthetic valve may be configured similarly as embodiments of prosthetic valves disclosed herein and may include one or more prosthetic valve leaflets and may be configured to be deployed to a native valve. Other configurations of prosthetic valves may be utilized as desired. The surface roughness of one or more prosthetic valve leaflets 160 may be varied or the surface roughness of another portion of a prosthetic valve such as a fabric, an anchor, a sealing body, or a sealing skirt, among other portions, may be varied. For example, a sealing body 11 as shown in FIG. 1 including a sealing skirt 24 may have a surface roughness altered. One or more of the anchors 17 may have a surface roughness altered as desired, including a fabric applied to the one or more anchors 17. Other portions may have a surface roughness altered.

In embodiments herein, one or more micropatterns may be applied to a portion of a prosthetic valve to alter the surface roughness. The one or more micropatterns may be applied to a prosthetic valve leaflet 160 or another portion of a prosthetic valve such as a fabric, an anchor, a sealing body, or a sealing skirt, among other portions.

Micropatterns may be applied to a prosthetic valve leaflet 160 or another portion of a prosthetic valve in a variety of manners, including laser milling, and other methods of forming micropatterns. Micropatterns may be applied to a surface of a prosthetic valve leaflet 160 or a surface of another portion of a prosthetic valve.

The micropatterns may be applied to create a pattern on the prosthetic valve leaflet 160 or other portion of a prosthetic valve, or in embodiments a similar method may be utilized to smooth a surface of a prosthetic valve leaflet 160 or a surface of another portion of a prosthetic valve (e.g., a fabric, an anchor, a sealing body, or a sealing skirt).

For example, laser milling may be applied to smooth one or more surfaces (such as an inward facing surface and an outward facing surface) of a prosthetic valve leaflet 160. An inward facing surface may be a surface that faces the flow channel of the prosthetic valve, and an outward facing surface may be a surface that faces away from the flow channel of the prosthetic valve. Either or both surfaces may be smoothed in embodiments herein. Smoothing, which may be via laser milling or another method, may be applied to one or more surfaces of portions of a prosthetic valve, such as a sealing body, a sealing skirt, or a fabric, among other portions.

In embodiments, applying a micropattern may include applying a pattern to the prosthetic valve leaflet, or other portion of the prosthetic valve, with the pattern having a height. The micropattern may be on a micrometer length scale according to embodiments, or on another scale as desired. The micropatterns may be applied to alter a surface roughness of at least a portion of a prosthetic valve.

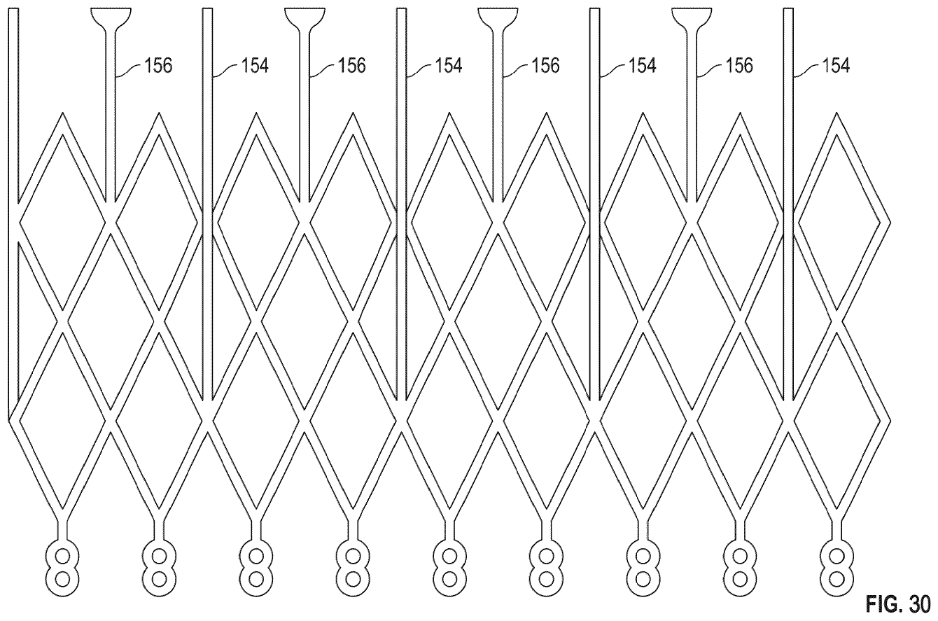

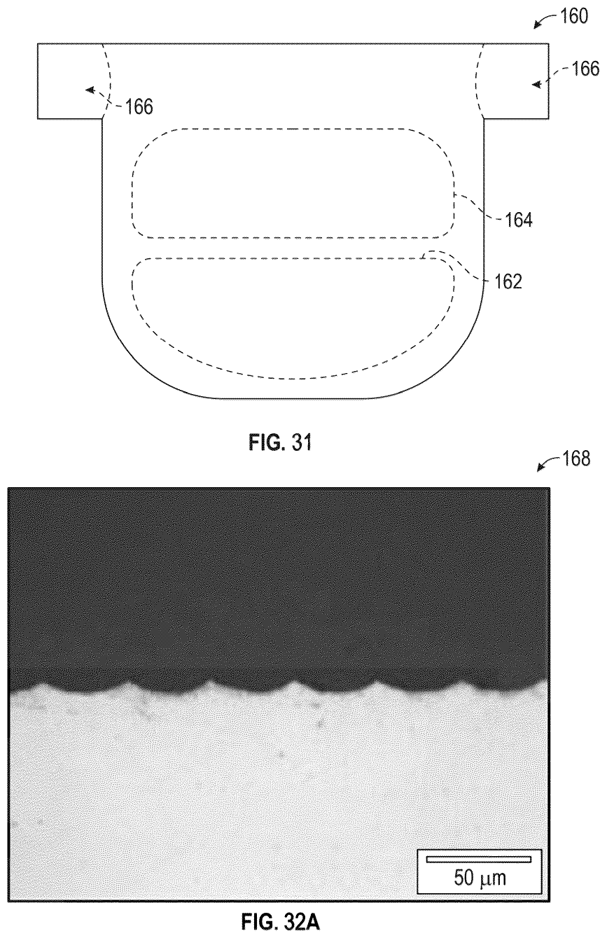

FIG. 31 illustrates an embodiment of a prosthetic valve leaflet 160 that may be utilized with a prosthetic valve and may include one or more micropatterns applied to the prosthetic valve leaflet 160. The prosthetic valve leaflet 160, for example, may include multiple portions 162, 164, 166 that may each include a different or same configuration of micropattern applied to that portion of the prosthetic valve leaflet 160. In embodiments, a micropattern may be applied that may alter a surface roughness of the prosthetic valve leaflet 160.

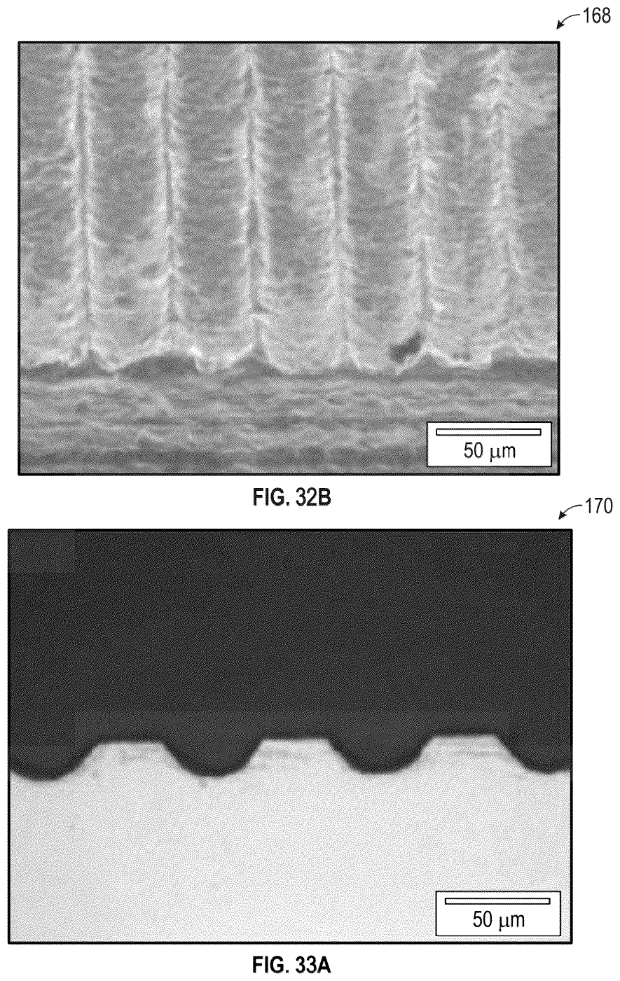

FIG. 32A, for example, illustrates a micropattern 168 according to embodiments herein having a plurality of peaks separated by troughs. The micropattern 168 may be applied to a prosthetic valve leaflet or another portion of a prosthetic valve (e.g., a fabric, an anchor, a sealing body, or a sealing skirt). The peaks may have a height on a micrometer scale. FIG. 32B illustrates the peaks may form channels on a surface of a prosthetic valve leaflet 160 or another surface of a portion of a prosthetic valve.

FIG. 33A illustrates a micropattern 170 according to embodiments herein in which the peaks include plateau portions. The micropattern 170 may be applied to a prosthetic valve leaflet or another portion of a prosthetic valve (e.g., a fabric, an anchor, a sealing body, or a sealing skirt). The plateau portions may be separated by troughs. FIG. 33B illustrates the peaks may form channels on the surface of a prosthetic valve leaflet 160 or another surface of a portion of a prosthetic valve. The peaks may have a height on a micrometer scale.

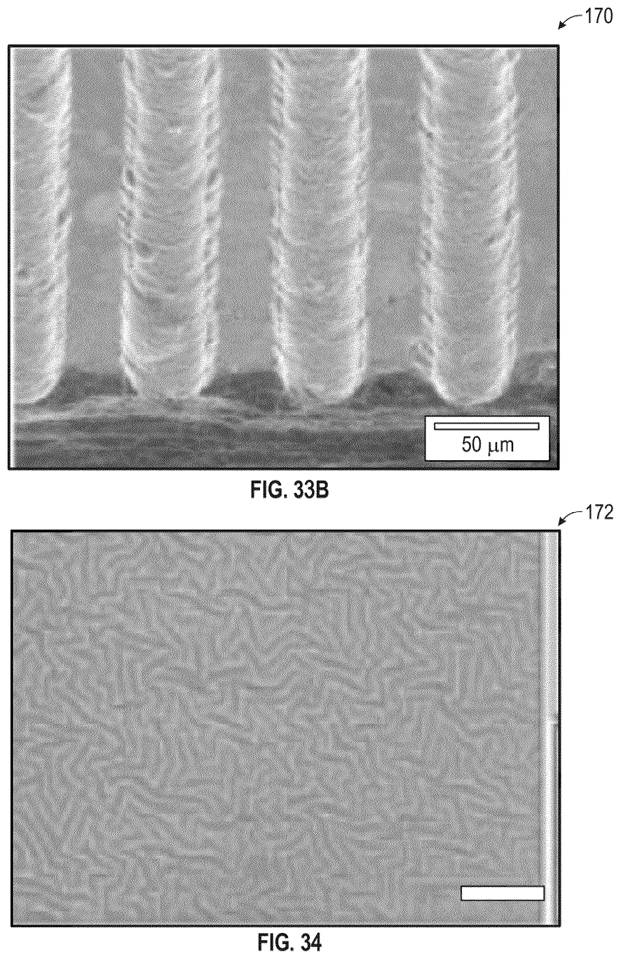

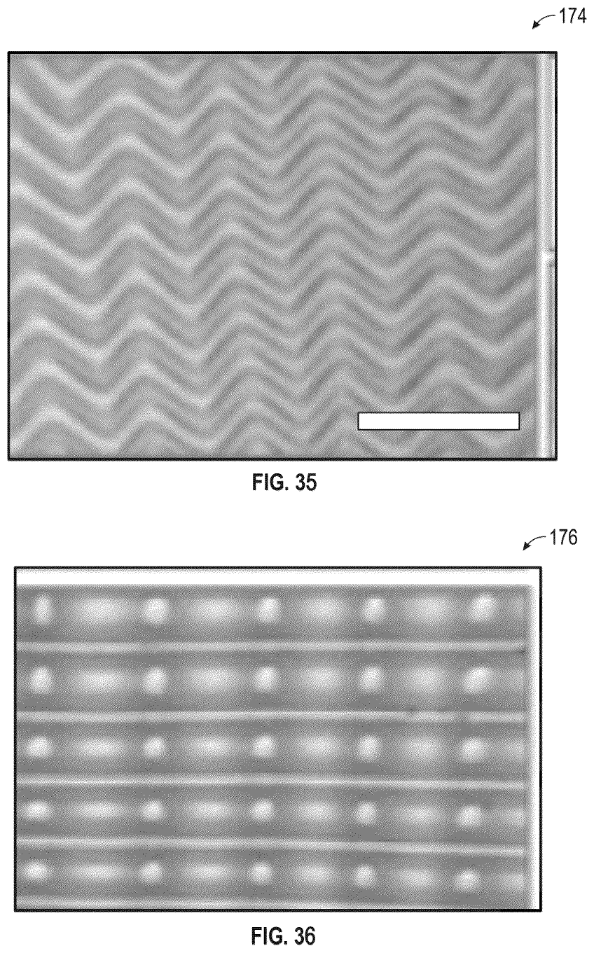

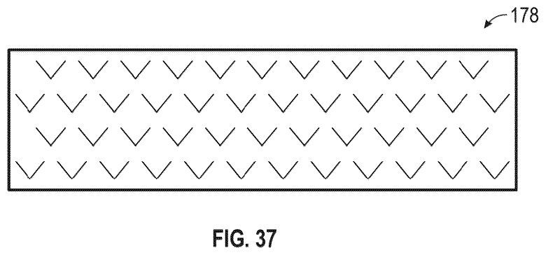

Various patterns may be formed on the prosthetic valve leaflet 160 or another portion of a prosthetic valve. FIG. 34 illustrates a non-uniform pattern 172 having a scattered appearance. FIG. 35 illustrates a sharklet pattern 174 that is uniform and may have an appearance of a plurality of chevrons. FIG. 36 illustrates a uniform checkered pattern 176. FIG. 37 illustrates another form of sharklet pattern 178 having an appearance of a plurality of chevrons. Other patterns may include a hexagonal pattern for sealing and providing wet adhesion. Various patterns may be applied as desired.

Different configurations of patterns may be applied to different portions of the prosthetic valve leaflet 160 or another portion of a prosthetic valve as desired. For example, a micropattern may include one or more of a checkered pattern or a sharklet pattern. The portions may be on an inward facing surface of the prosthetic valve leaflet, or an outward facing surface, or another surface, such as a surface that couples to the valve frame. A portion of the prosthetic valve leaflet may have a first configuration of the micropattern applied to it, and another portion of the prosthetic valve leaflet may have a second configuration applied to it that is different than the first configuration.

Referring to FIG. 31, exemplary portions of a prosthetic valve leaflet 160 may comprise a portion 162 that contacts another similar portion of another leaflet during coaptation of the leaflets. The portions may comprise a portion 164 that fluid (e.g., blood) flows along during opening of the leaflets. The portions may comprise a portion 166 may couples to the valve frame. Various other portions may include a micropattern.

The configuration of the micropattern applied may be selected to provide a desired performance of the portion of the prosthetic valve leaflet 160. For example, a portion 162 that contacts another portion of a leaflet during coaptation may include a pattern that increases or reduces friction with another portion of a leaflet. A portion 164 that fluid flows along may include a pattern that enhances fluid flow. A portion 166 that couples to the valve frame may include a pattern that enhances friction to improve grip to the valve frame. Micropatterns may be applied to provide results that include but are not limited to reducing formation of a thrombus on the prosthetic valve leaflet, enhancing biocompatibility of the prosthetic valve leaflet with the patient's body, altering fluid flow through the prosthetic valve, improving coaptation between the plurality of prosthetic valve leaflets, and improving coupling of the prosthetic valve leaflet to the frame. Various other results may be produced by applying a micropattern to one or more portions of a prosthetic valve leaflet. Such results may be utilized with a prosthetic valve leaflet, or may be utilized with another portion of a prosthetic valve, such as a sealing body, an anchor, a sealing skirt, or a fabric. For example, a surface of a sealing body, an anchor, a sealing skirt, or a fabric, such as an outer surface or other surface may include a micropattern that may include reducing formation of a thrombus on the portion of the prosthetic valve, enhancing biocompatibility of the portion of the prosthetic valve with the patient's body, and altering fluid flow through the prosthetic valve, among other results.

At least one of the one or more prosthetic valve leaflets may be coupled to a frame, as disclosed herein. The frame may be configured to support the plurality of prosthetic valve leaflets. At least one of the prosthetic valve leaflets may have a micropattern applied to the prosthetic valve leaflet. The resulting prosthetic valve, for example, may comprise any of the prosthetic valves disclosed herein or another form of prosthetic valve. The prosthetic valve may be deployed to a native valve.

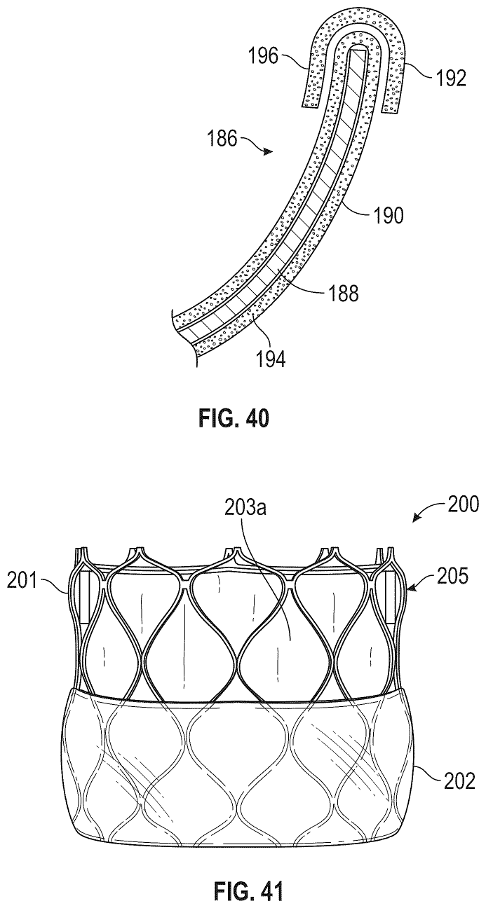

In embodiments, the micropatterns disclosed herein may be applied to another portion of a prosthetic valve. A prosthetic valve configured to be deployed to a native valve of a body may be provided, the prosthetic valve including one or more prosthetic valve leaflets and including a micropattern applied to at least a portion of the prosthetic valve. For example, one or more portions of a sealing body, an anchor, a sealing skirt, or a fabric as disclosed herein may have a micropattern applied to it, among other portions of a prosthetic valve. At least a portion of a prosthetic valve may comprise a fabric as disclosed herein, and a micropattern may be applied a fabric. The fabric may be a fabric of a sealing body or sealing skirt, among other forms of fabric. The fabric may comprise fabric on one or more anchors (such as distal anchors or other forms of anchors) or a prosthetic valve. An outer surface of such a portion may include the micropattern, among other surfaces. An outer surface 179 of a sealing body, fabric, and sealing skirt that may include a micropattern is marked in FIG. 1, for example. Fabric of an anchor that may include a micropattern is shown in FIGS. 39 and 40, for example.

A micropattern that may be applied to a portion of a prosthetic valve may comprise any configuration of micropattern as desired. For example, a pattern may be utilized to enhance friction and thus sealing between an outer surface of a sealing skirt and a surface of a valve annulus. Enhanced fixation between the native annulus and a sealing skirt may result. Such a pattern may comprise the sharklet pattern 178 as shown in FIG. 37. A sharklet pattern, for example, may produce a hard point that may enhance adherence between the sealing skirt and the native valve annulus. One or more of a checkered pattern or a sharklet pattern may be utilized. Other forms of patterns may be utilized as desired.

The embodiments of micropatterns may provide a variety of benefits. Various modifications of the embodiments may be provided, including substitution or addition of features across various embodiments disclosed herein. The embodiments of micropatterns may be utilized solely or in combination with features of other embodiments disclosed herein.

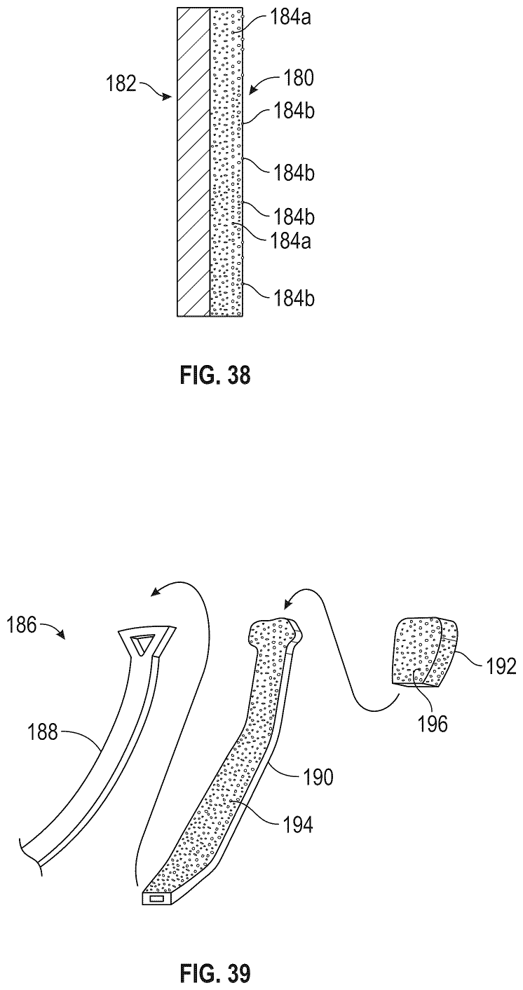

FIGS. 38-44 illustrate embodiments in which one or more microbeads may be utilized. The microbeads may be utilized with a prosthetic valve configured to be deployed to a native valve of a body. In embodiments, the one or more microbeads may be for emitting a substance into the body.

The microbeads may each be configured to emit the substance into the body by diffusion of the microbeads. For example, the microbeads may be made of the substance or coated with the substance and configured to emit the substance through a gradual or slow diffusion of the substance into the body. The microbeads accordingly may gradually decrease in size as the emission of the substance occurs. Other methods of emission by the microbeads may be utilized as desired.

The substance that may be emitted by the microbeads may comprise a variety of forms of substances, including but not limited to a drug. The drug may comprise a chemical that may be emitted into the body in a liquid form, although other forms of emission may be utilized as desired. The substance that may be emitted by the microbeads may be configured to produce a therapeutic effect for the patient's body, although other forms of substances may be emitted by the microbeads as desired.

The drug may comprise an anti-thrombus drug in embodiments. The anti-thrombus drug may comprise a vitamin K antagonist, anticoagulants (including a novel oral anticoagulant (NOAC) or a non-VKA oral anticoagulant (NOAC) or a direct-acting oral anticoagulant (DOAC)), among other forms of anti-thrombus drugs. In embodiments, other forms of drugs may be utilized as desired.

The drug may be emitted to reduce the formation of a thrombus on a prosthetic leaflet or otherwise upon the prosthetic valve. The drug, for example, may reduce accumulation of thrombus on a prosthetic leaflet or otherwise upon the prosthetic valve. The drug accordingly may be emitted in embodiments to reduce prosthetic leaflet thickening or reduce thickening of other portions of the prosthetic valve in embodiments.

The drug may be emitted proximate the surface for reduction of thrombus (e.g., a prosthetic valve leaflet or another portion of the prosthetic valve). As such, the one or more microbeads may be positioned proximate such a surface and may emit the drug or other substance proximate the surface. The one or more microbeads in embodiments may emit the substance in a localized manner within the patient's body proximate the surfaces for reduction of thrombus. The one or more microbeads may diffuse into the bloodstream local to the prosthetic valve to reduce the accumulation of thrombus.

In embodiments, the one or more microbeads may be positioned on a fabric or skirt of the prosthetic valve. The fabric or skirt may comprise a portion of a sealing body configured to contact a portion of an annulus of a native valve. For example, FIG. 38 illustrates a cross sectional detail view of a portion of a sealing skirt 180 that may be positioned on an outer frame 182. The sealing skirt 180 may be configured similarly as the sealing skirt 24 shown in FIG. 5, and the outer frame 182 may be configured similarly as the outer frame 20 shown in FIG. 5. The sealing skirt 180 may comprise a fabric or other form of material. Other portions of a prosthetic valve may include a sealing skirt 180 or fabric or may include one or more microbeads. The microbeads may be positioned on a portion of a sealing body or other portion of a prosthetic valve that may lack a fabric or skirt. The microbeads may be positioned on a frame or other portion of a prosthetic valve for example. The prosthetic valve may be configured similarly as other prosthetic valves disclosed herein and may include one or more prosthetic valve leaflets or other features of prosthetic valves disclosed herein. Other forms of prosthetic valves may be utilized as desired.

Referring to FIG. 38, the one or more microbeads may be embedded into the sealing skirt 180. For example, microbeads 184a may be wholly embedded with the sealing skirt 180 and within a fabric of the sealing skirt 180. As such, upon fluid (e.g., blood) contact with the microbeads 184a, the microbeads 184a may diffuse and emit a substance into the patient's body, which may be proximate the native heart valve and the components of the prosthetic heart valve. The microbeads 184a may emit the substance local to components of the prosthetic heart valve to reduce formation of thrombus on such portions.

In embodiments, microbeads 184b may be partially embedded into the sealing skirt 180 and the fabric of the sealing skirt 180, or positioned on an exterior surface of the sealing skirt 180 and fabric of the sealing skirt. Such microbeads 184b may be partially or wholly exposed exterior of the sealing skirt 180. The exterior surface may comprise an outer surface 179 as marked in FIG. 1 for example.

In embodiments, the varied depth of the microbeads 184a, 184b from the exterior surface of the sealing skirt 180 may produce a gradual release of the substance into the patient's body. For example, the microbeads 184b at the exterior surface of the sealing skirt 180 may emit or diffuse the substance more quickly than the microbeads 184a wholly embedded in the sealing skirt 180. As such, a gradual rate of emission of the substance from the microbeads 184a, 184b may occur based on the size, number, and/or position of the microbeads from the exterior surface of the sealing skirt 180. The rate of emission may be controlled based on controlling the size, number, and/or position of the microbeads from the exterior surface of the sealing skirt 180 in embodiments.

The microbeads may be positioned in other locations relative to the prosthetic valve. The locations may comprise fabric or another portion of the prosthetic valve. FIG. 39, for example, illustrates an assembly view of fabric or other material that may be positioned on an anchor of a prosthetic valve. The prosthetic valve may include one or more anchors, and the one or more microbeads may be positioned on the one or more anchors. The anchors may comprise a distal anchor in embodiments. A distal anchor 186, for example, may include an anchor arm 188 that may be covered with a sleeve 190, which may be covered with an end cover 192.

FIG. 40 illustrates a side cross sectional view of the distal anchor 186, showing the sleeve 190 extending over the anchor arm 188 and the end cover 192 over the sleeve 190. The resulting configuration of the distal anchor 186 may have an appearance similar to the anchors 17 shown in FIG. 1. A distal anchor 186 may be configured to hook around a leaflet of the native valve or may have another configuration as desired.

The sleeve 190 and the end cover 192 may comprise fabric that may include the one or more microbeads 194,196. The microbeads 194,196 may be configured similarly as the microbeads 184a, b, yet may be positioned on the respective sleeve 190 and end cover 192. The microbeads 194, 196 may be configured to emit a substance in a similar manner as the microbeads 184a, b, and the substance may be configured to reduce formation of thrombus or may produce another result as desired.

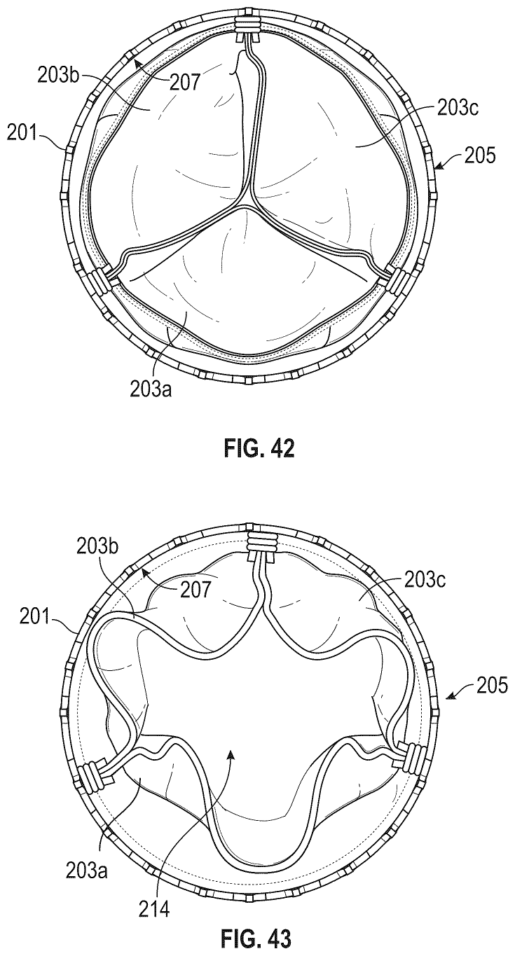

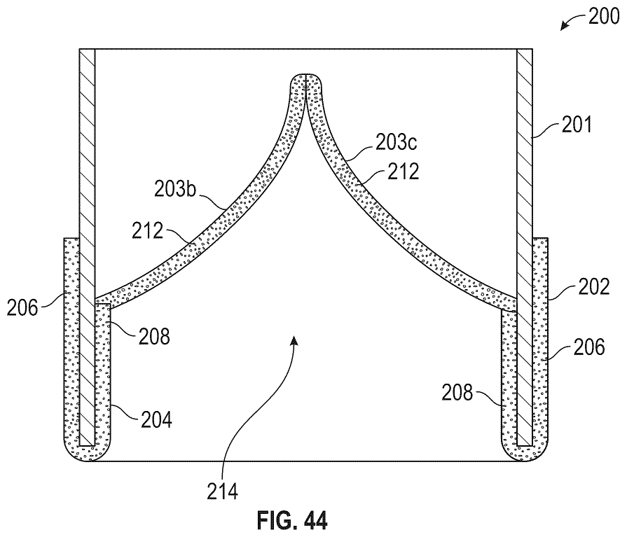

The components discussed with respect to FIGS. 38-40 may comprise components of a prosthetic valve configured to be deployed to a mitral valve or a tricuspid valve of a body. However, the use of the microbeads is not limited to such a prosthetic valve, and may be utilized in other forms of prosthetic valves. FIGS. 41-43, for example, illustrate views of a prosthetic valve 200 configured to be deployed to an aortic valve of a body. The prosthetic valve 200, for example, may include a frame 201 supporting one or more prosthetic valve leaflets 203a-c and including an outer surface 205 and an inner surface 207 (marked in FIG. 42). The prosthetic valve leaflets 203a-c may be positioned within a flow channel 214 (marked in FIG. 43) of the prosthetic valve 200. The prosthetic valve 200 may include a skirt 202 that may be made of a fabric. The skirt 202 may comprise an outer surface of the prosthetic valve 200 and may be configured to contact a portion of the patient's body (e.g., native valve leaflets and/or an annulus of the native aortic valve). In embodiments, an inner surface of the prosthetic valve 200 may include a skirt 204 (as marked in FIG. 44).

FIG. 44 illustrates a cross sectional schematic view of the prosthetic valve 200. The skirt 202 may include one or more microbeads 206, which may be embedded in the skirt 202 in a similar manner as the microbeads 184a, b, discussed with respect to FIG. 38. An interior skirt 204 may include one or more microbeads 208 which may be embedded in the skirt 204 in a similar manner as the microbeads 184a, b, discussed with respect to FIG. 38.

In embodiments, microbeads 212 may be positioned on the prosthetic valve leaflets 203a— c. The prosthetic valve leaflets 203a—c may include microbeads 212 that may be configured similarly as the microbeads 184a, b. The microbeads 206, 208, 212 may each be configured to emit a substance that may be configured to provide a therapeutic effect for the patient's body. For example, a drug may be emitted that may reduce formation of thrombus or may produce another result as desired. The reduction of thrombus may be on the prosthetic valve leaflets 203a—c or another portion of the prosthetic valve 200. The microbeads 212 on the prosthetic valve leaflets 203a—c may be configured to reduce the production of thrombus on the prosthetic valve leaflets 203a—c. The microbeads 206, 208 may be configured to reduce production of thrombus on the prosthetic valve leaflets 203a—c or another portion of the prosthetic valve 200 (e.g., an interior flow channel 214 of the prosthetic valve 200).

In embodiments, microbeads may be positioned on portions of a prosthetic valve that may not comprise a skirt or fabric. For example, portions of a frame or another portion of a valve body may have one or more microbeads coupled thereto. The microbeads may be configured to emit a substance in a similar manner as other microbeads disclosed herein.

Various modifications of the embodiments may be provided, including substitution or addition of features across various embodiments disclosed herein. One or more microbeads may be utilized solely or in combination with features of other embodiments disclosed herein.

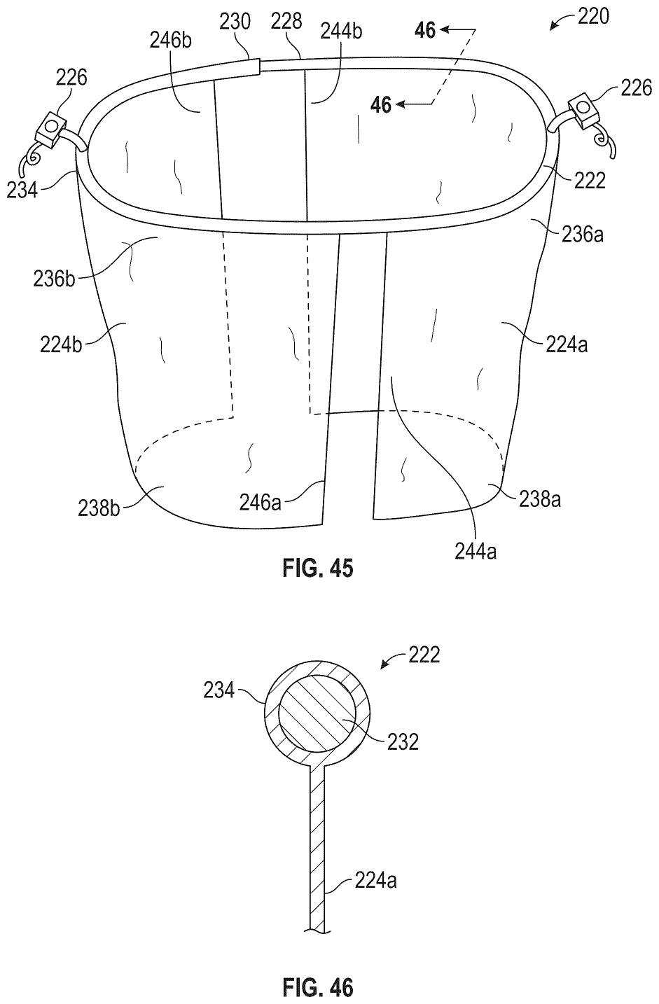

FIGS. 45-55 illustrate embodiments of a prosthetic valve that may be utilized herein. Referring to FIG. 45, the prosthetic valve 220 may include a support ring 222, one or more prosthetic valve leaflets 224a, b, and in embodiments may include one or more anchors 226.

The support ring 222 may be configured to extend about an annulus of the native valve. The support ring 222 may have an annular shape and may include a first end portion 228 and a second end portion 230 that may be coupled together to form a closed loop for the support ring 222. In embodiments, the support ring 222 may comprise an open ring with a gap between the end portions 228, 230, or may have another configuration as desired.

FIG. 46 illustrates a cross sectional view of the support ring 222 along line 46-46 in FIG. 45. The support ring 222 may include a ring body 232, which may comprise an inner ring body, and may include a sheath 234 extending over the ring body 232. The support ring 222 and ring body 232 may have a circular cross sectional shape, or may have another shape (e.g., rectangular, triangular, or other cross sectional shape) as desired. The support ring 222 may be flexible and may be configured to form into a desired shape upon deployment. For example, the support ring 222 may be configured to conform to a shape of an annulus upon being deployed. The shape may comprise an ovoid shape or a “D” shape, among other possible shapes. The support ring 222 upon deployment, accordingly may have the shape of a circle or may have an elongate shape such as an ovoid shape or other shape.

The support ring 222 may be configured to be expanded from an undeployed configuration to a deployed configuration. For example, the flexibility of the support ring 222 may allow the support ring 222 and the prosthetic valve 220 to be compressed into an undeployed, unexpanded, or linearized configuration and then expanded to a deployed or expanded configuration. The support ring 222, for example, may be folded to be placed in an undeployed, unexpanded, or linearized configuration and then expanded to form a ring shape as shown in FIG. 45 for example.

The ring body 232, in embodiments, may comprise a body made of metal, a polymer, or an alloy material that may be configured to flex. The ring body 232, for example, may comprise a shape memory material such as Nitinol or another form of shape memory material. The ring body 232 may be made of a material such that upon deployment, the ring body 232 may automatically expand into a deployed configuration such as a ring shape that may conform to a shape of an annulus. Other materials may be utilized in embodiments.

The sheath 234 may extend over the ring body 232 and may comprise a coupler to the one or more prosthetic valve leaflets 224a, 224b. The one or more prosthetic valve leaflets 224a, 224b may be coupled to the support ring 222 via the sheath 234. The sheath 234 may extend over at least a portion of the support ring 222 and may extend fully or partially over the ring body 232. The sheath 234 may be integrally coupled to the one or more prosthetic valve leaflets 224a, 224b to couple the leaflets 224a, 224b to the ring body 232. The sheath 234 may extend along the length or perimeter of the ring body 232 and may extend over the entire length or perimeter or over only a portion of the length or perimeter. In embodiments, other forms of couplers may be utilized to couple the one or more prosthetic valve leaflets 224a, 224b to the ring body 232 and accordingly to the support ring 222.

Referring to FIG. 45, the one or more prosthetic valve leaflets 224a, b may each include a respective first end portion 236a, b and a second end portion 238a, b extending in a distal direction from the first end portion 236a, b. The first end portions 236a, b may be coupled to the support ring 222 and the second end portions 238a, b may extend distally from the first end portions 236a, b. The first end portions 236a, b may couple to the ring body 232 via the sheath 234 in embodiments, and the second end portions 238a, b may comprise free ends of the leaflets 224a, b configured to move inward and outward to mimic operation of the native valve leaflets. The second end portions 238a, b may be uncoupled to other portions of the prosthetic valve 220 and other structures of the prosthetic valve 220 may not surround or restrain the second end portions 238a, b. As such, the second end portions 238a, b, may be configured to contact the native valve leaflets and may move along with the native valve leaflets upon deployment. The first end portions 236a, b may comprise supported ends of the prosthetic valve leaflets and the second end portions 238a, b may comprise unsupported ends of the prosthetic valve leaflets.

The prosthetic valve leaflets may extend distally from the support ring 222 to form a cylindrical curtain structure as shown in FIG. 45. The first end portions 236a, b may have a cylindrical shape that may match a shape of the support ring 222. The cylindrical curtain structure may extend distally from the support ring 222. The second end portions 238a, b, may be configured to move towards and away from each other upon the opening and closing of the prosthetic leaflets of the prosthetic valve 220.

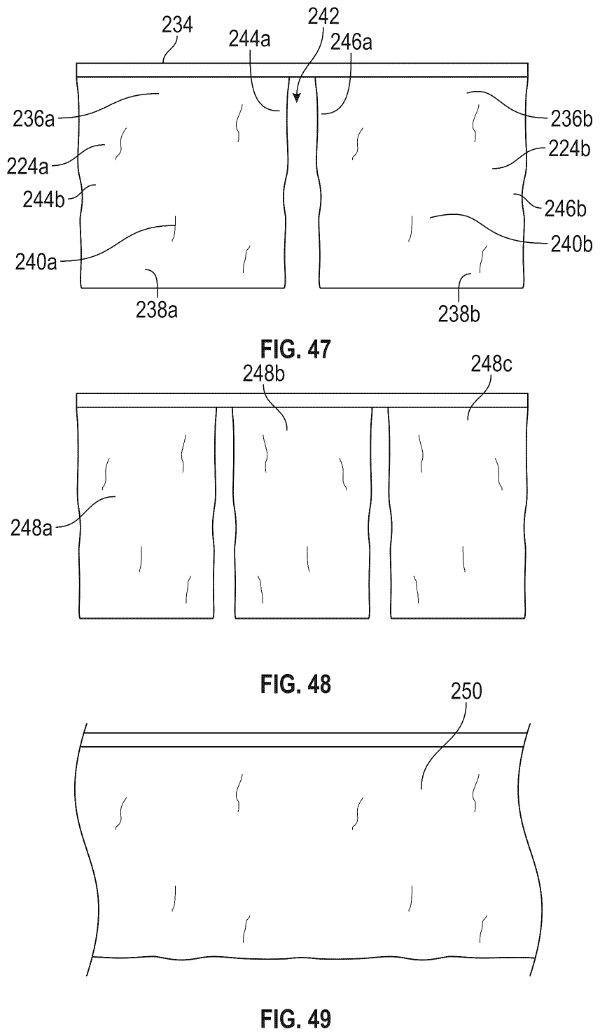

FIG. 47 illustrates a view of the prosthetic valve leaflets 224a, b flattened. The prosthetic valve leaflets 224a, b may extend from the respective first end portions 236a, b to the second end portions 238a, b and may have a rectangular shape when flattened or another shape as desired. A central portion 240a, b of the respective prosthetic valve leaflets 224a, b may be positioned between the respective first end portions 236a, b and the second end portions 238a, b and may coapt during opening and closing of the prosthetic valve leaflets 224a, b. The prosthetic valve leaflets 224a, b may include a gap 242 positioned circumferentially between adjacent edges 244a and 246a and may include a gap between adjacent edges 244b, 246b. In embodiments, one or more of the gaps may be excluded.

In embodiments, a greater or lesser number of prosthetic valve leaflets may be utilized. In embodiments, at least two prosthetic valve leaflets may be utilized. The prosthetic valve leaflets may be circumferentially spaced from each other. FIG. 48, for example, illustrates a flattened configuration in which three prosthetic valve leaflets 248a, b, c, may be utilized. Such a configuration may be utilized in an embodiment in which the prosthetic valve is deployed to a tricuspid valve, for example. FIG. 49 illustrates a flattened configuration in which a single prosthetic valve leaflet 250 may be utilized. The prosthetic valve leaflet 250 may have end portions that may meet or overlap, or in embodiments, may comprise a unitary body extending from the support ring 222.

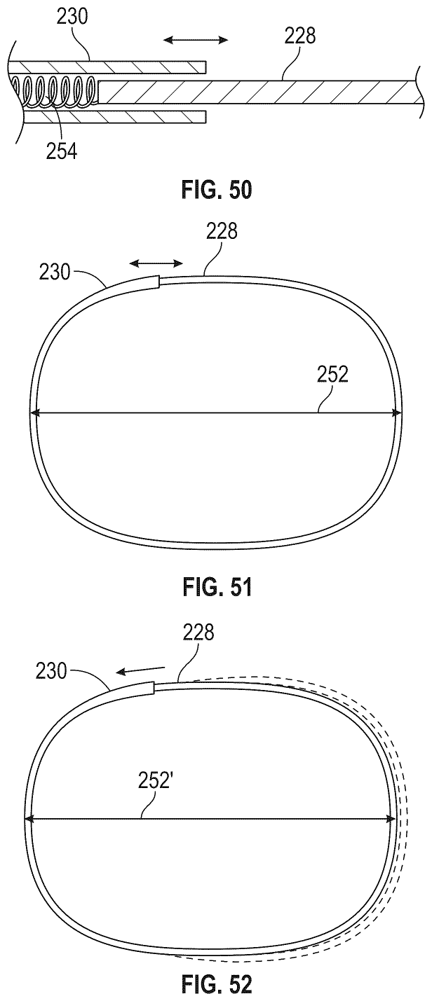

In embodiments, the first end portion 228 and the second end portion 230 of the support ring 222 may be configured to move relative to each other. For example, referring to FIG. 50, the first end portion 228 may be configured to insert into the second end portion 230, and the second end portion 230 may slide relative to the first end portion 228. The second end portion 230 may slide relative to the first end portion 228 to allow the support ring 222 to move. The movement of the second end portion 230 relative to the first end portion 228 may be either into and towards the first end portion 228 or out of and away from the first end portion 228. The movement may allow a size and shape of the support ring 222 to vary. For example, referring to FIG. 51, upon deployment, the support ring 222 may have a diameter 252. The second end portion 230 may slide relative to the first end portion 228 to vary a size of the diameter 252 of the support ring 222. Upon the first end portion 228 moving into and towards the second end portion 230, the diameter 252 may reduce to a diameter 252′ shown in FIG. 52. The movement of the first end portion 228 relative to the second end portion 230 may accordingly allow the size of the support ring 222 to vary.

Referring to FIG. 50, in embodiments, a spring 254 may be provided that may bias the first end portion 228 relative to the second end portion 230. The spring 254 may be biased to draw the first end portion 228 into and towards the second end portion 230. As such, the spring 254 may be configured to reduce the size of the support ring 222 and may be configured to reduce the size of an annulus that the support ring 222 is coupled to. In embodiments, the spring 254 may be biased to move the first end portion 228 away from the second end portion 230. As such, in embodiments, the spring 254 may encourage an expansion of the support ring 222 upon deployment, which may be overcome by the size of the annulus reducing due to the cardiac cycle or upon another reduction of the size of the annulus.

In embodiments, the support ring 222 may be configured to vary in size to accommodate the movement of the annulus during the cardiac cycle. In embodiments, the second end portion 230 may be configured to automatically slide relative to the first end portion 228 in response to a variation in a diameter of the annulus. The support ring 222 may be configured to vary in size to allow the support ring 222 to reduce in size along with the annulus. For example, the size of the annulus may reduce as a result of the therapeutic benefits provided by the prosthetic valve 220, or as a result of reduced size of the annulus produced by other effects. The annulus may reduce in size, for example, as stress upon the native valve is reduced and/or stress producing dilation in a ventricle or atrium is reduced. The support ring 222 may reduce in size along with the annulus. In embodiments, the support ring 222 may increase in size if further dilation of the annulus occurs as a result of maladies or other causes of dilation of the annulus.

Referring to FIG. 45, in embodiments, one or more anchors 226 may be provided. The anchors 226 may be configured to anchor the support ring 222 to the native valve. The anchors may have a variety of forms and may comprise a penetrating body such as a screw, a hook, a barb, a clamp, or a clasp, among other forms of anchors. The anchors 226 may each be configured to penetrate into tissue. The anchors 226 may be coupled to the support ring 222 and may be coupled to the ring body 232 through the sheath 234 for example. Other configurations of anchors may be utilized in embodiments as desired.

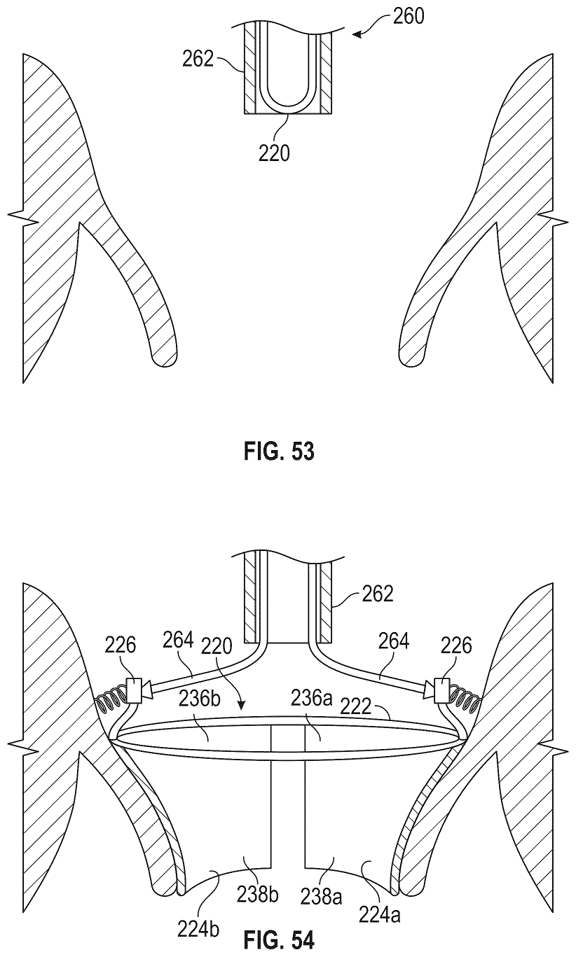

FIGS. 53-55 illustrate an exemplary deployment sequence for the prosthetic valve 220. FIG. 53, for example, illustrates the prosthetic valve 220 in an undeployed configuration, in which it may be positioned within a delivery system 260. The delivery system 260, for example, may include an implant retention area in the form of a capsule 262 that retains the prosthetic valve 220. The prosthetic valve 220, and particularly the support ring 222 may be in a compressed configuration, which may comprise an elongated configuration in which the support ring 222 has a reduced thickness and increased length from the deployed configuration as shown in FIG. 51 for example. The support ring 222 may be elongated and may be folded in the undeployed configuration. The support ring 222 may have an elongate shape with sides of the support ring 222 drawn together in a compressed state.

FIG. 54 illustrate the prosthetic valve 220 having been deployed from the capsule 262. The support ring 222 may deploy and expand outward upon being released from the capsule 262. Such expansion may occur automatically in embodiments, or the support ring 222 may be expanded via an expansion device or another method of expansion. The support ring 222 may expand to fit the shape of the annulus as desired.

The second end portions 238a, b of the prosthetic valve leaflets 224a, b may be configured to extend from the first end portion 236a, b in a direction towards the ventricle.

The prosthetic valve leaflets 224a, b may contact and overlay at least a portion of the native valve leaflets. The prosthetic valve leaflets 224a, b accordingly may be configured to move with an opening and closing operation of the native valve leaflets. In embodiments, the prosthetic valve leaflets 224a, b may open and close independent of the native valve leaflets.

The anchors 226 are shown in FIG. 54 not yet inserted into the tissue of the patient's body. Deployment devices 264 of the delivery system 260 may engage the anchors 226 and may be utilized to drive the anchors 226 into the tissue to anchor the anchors 226 into position.

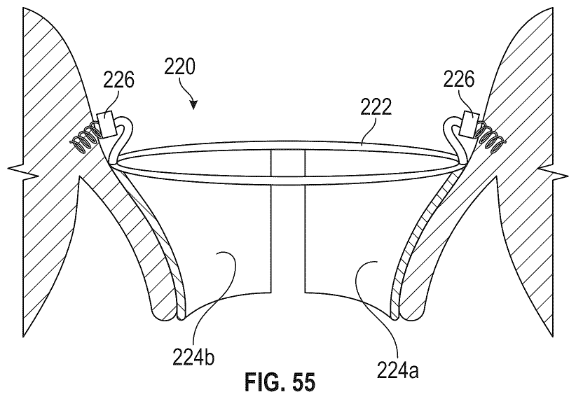

FIG. 55, for example, illustrates the anchors 226 inserted into position to anchor the support ring 222 to the native valve.

Various other methods of deployment of the prosthetic valve 220 may be utilized as desired.

The prosthetic valve 220 may comprise a valve having a single support body (e.g., the support ring 222) for the prosthetic valve leaflets and may lack a frame in embodiments. The lack of a frame may reduce the complexity of the prosthetic valve 220 and may reduce the overall material that may be deployed into the patient's body upon delivery of the prosthetic valve 220. The prosthetic valve 220 accordingly in embodiments may comprise a frameless prosthetic valve 220. In embodiments, the prosthetic valve 220 may be limited to the components disclosed herein and may lack additional components. In embodiments, additional components may be utilized as desired.

Various modifications of the embodiments may be provided, including substitution or addition of features across various embodiments disclosed herein. The embodiments of FIGS. 45-55 may be utilized solely or in combination with features of other embodiments disclosed herein.

The embodiments of prosthetic valves may be utilized in a mitral valve as disclosed herein or may be utilized in other deployment locations such as a native tricuspid valve, or other deployment locations. Deployment to aortic or pulmonary valves, or other implantation sites may be utilized.

Various modifications of the embodiments disclosed herein may be provided. Features of embodiments may be modified, substituted, excluded, or combined across embodiments as desired. Combinations of features across embodiments may be provided as desired. Combinations of features may be provided across embodiments with other features of such embodiments being excluded if desired.

The various embodiments of sealing skirts disclosed herein may have a variety of forms, including cloth skirts, foam skirts, or braided skirts as desired. Various materials may be utilized as desired.

The implants disclosed herein may include prosthetic heart valves or other forms of implants, such as stents or filters, or diagnostic devices, among others. The implants may be expandable implants configured to move from a compressed or undeployed state to an expanded or deployed state. The implants may be compressible implants configured to be compressed inward to have a reduced outer profile and to move the implant to the compressed or undeployed state.

Various forms of delivery apparatuses may be utilized with the embodiments disclosed herein. The delivery apparatuses as disclosed herein may be utilized for aortic, mitral, tricuspid, and pulmonary replacement and repair as well. The delivery apparatuses may comprise delivery apparatuses for delivery of other forms of implants, such as stents or filters, or diagnostic devices, among others.

The implants and the systems disclosed herein may be used in transcatheter mitral or tricuspid implantation, as well as aortic valve implantation (TAVI) or replacement of other native heart valves (e.g., pulmonary valves). The delivery apparatuses and the systems disclosed herein may be utilized for transarterial access, including transfemoral access, to a patient's heart. The delivery apparatuses and systems may be utilized in transcatheter percutaneous procedures, including transarterial procedures, which may be transfemoral or transjugular. Transapical procedures, among others, may also be utilized. Other procedures may be utilized as desired.

In addition, the methods herein are not limited to the methods specifically described, and may include methods of utilizing the systems and apparatuses disclosed herein. The steps of the methods may be modified, excluded, or added to, with systems, apparatuses, and methods disclosed herein. The embodiments disclosed herein may comprise systems for implantation within a human body in embodiments.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of systems, apparatuses, and methods as disclosed herein, which is defined solely by the claims. Accordingly, the systems, apparatuses, and methods are not limited to that precisely as shown and described.

Certain embodiments of systems, apparatuses, and methods are described herein, including the best mode known to the inventors for carrying out the same. Of course, variations on these described embodiments will become apparent to those of ordinary skill in the art upon reading the foregoing description. The inventor expects skilled artisans to employ such variations as appropriate, and the inventors intend for the systems, apparatuses, and methods to be practiced otherwise than specifically described herein. Accordingly, the systems, apparatuses, and methods include all modifications and equivalents of the subject matter recited in the claims appended hereto as permitted by applicable law. Moreover, any combination of the above-described embodiments in all possible variations thereof is encompassed by the systems, apparatuses, and methods unless otherwise indicated herein or otherwise clearly contradicted by context.

Groupings of alternative embodiments, elements, or steps of the systems, apparatuses, and methods are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses an approximation that may vary, yet is capable of performing the desired operation or process discussed herein.

The terms “a,” “an,” “the” and similar referents used in the context of describing the systems, apparatuses, and methods (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the systems, apparatuses, and methods and does not pose a limitation on the scope of the systems, apparatuses, and methods otherwise claimed. No language in the present specification should be construed as indicating any non-claimed element essential to the practice of the systems, apparatuses, and methods.

All patents, patent publications, and other publications referenced and identified in the present specification are individually and expressly incorporated herein by reference in their entirety for the purpose of describing and disclosing, for example, the compositions and methodologies described in such publications that might be used in connection with the systems, apparatuses, and methods. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A system for replacing the function of a defective native heart valve, the system comprising:
a prosthetic heart valve including:
an inner frame having a proximal portion and a distal portion, the inner frame surrounding a flow channel, wherein the proximal portion comprises an inflow portion of the prosthetic heart valve and the distal portion comprises an outflow portion of the prosthetic heart valve,
an outer frame surrounding the inner frame, the outer frame having a proximal portion coupled to the proximal portion of the inner frame, wherein the outer frame includes a longitudinal hinge for allowing a first region of the outer frame to pivot relative to a second region of the outer frame, thereby allowing the outer frame to conform to a shape of an annulus of the defective native heart valve, and
a plurality of prosthetic valve leaflets positioned within the flow channel of the inner frame; and
a delivery catheter for delivering the prosthetic heart valve to the defective native heart valve.

2. The system of claim 1, wherein the longitudinal hinge allows for radially inward and radially outward rotation of the first region relative to the second region.

3. The system of claim 1, wherein the longitudinal hinge allows for rotation of the first region relative to the second region in only a single direction.

4. The system of claim 1, wherein the first region comprises a first axially extending column of the outer frame, and the second region comprises a second axially extending column of the outer frame that is adjacent to the first axially extending column.

5. The system of claim 4, wherein the first axially extending column includes a first strut cell of the outer frame, and the second axially extending column includes a second strut cell of the outer frame, and the longitudinal hinge couples a first strut of the first strut cell with a second strut of the second strut cell that is non-integral with the first strut.

6. The system of claim 1, wherein a clearance is provided between a distal portion of the outer frame and the inner frame.

7. The system of claim 1, wherein the prosthetic heart valve includes one or more distal anchors adapted to hook over one or more native leaflets of the defective native heart valve to anchor the prosthetic heart valve to the defective native heart valve.

8. The system of claim 1, further comprising a micropattern applied to at least a portion of the prosthetic heart valve.

9. The system of claim 1, wherein the prosthetic heart valve is a prosthetic mitral heart valve or a prosthetic tricuspid heart valve.

10. A prosthetic heart valve for replacing the function of a defective native heart valve, the prosthetic heart valve comprising:
an inner frame having a proximal portion and a distal portion, the inner frame surrounding a flow channel and a central axis of the prosthetic heart valve extending through the flow channel, wherein the proximal portion comprises an inflow portion of the prosthetic heart valve and the distal portion comprises an outflow portion of the prosthetic heart valve;
an outer frame having a proximal portion and a distal portion and surrounding the inner frame, the proximal portion of the outer frame being coupled to the proximal portion of the inner frame, and wherein the outer frame includes a first portion and a second portion and at least one rotation joint coupling the first portion to the second portion for the first portion to rotate relative to the second portion in a plane extending transverse to the central axis, to thereby allow the outer frame to conform to a shape of an annulus of the defective native heart valve; and
a plurality of prosthetic valve leaflets positioned within the flow channel and supported by the inner frame.

11. The prosthetic heart valve of claim 10, wherein the at least one rotation joint includes one or more of a suture joint, a t-bracket, a ball and socket joint, an overmolding, or a hinge.

12. The prosthetic heart valve of claim 11, wherein the suture joint includes a shear-lashing knot.

13. The prosthetic heart valve of claim 10, wherein the at least one rotation joint allows for radially inward and radially outward rotation of the first portion relative to the second portion.

14. The prosthetic heart valve of claim 10, wherein the at least one rotation joint allows for rotation of the first portion relative to the second portion in only a single direction.

15. The prosthetic heart valve of claim 10, wherein the first portion comprises a first axially extending column of the outer frame including a first strut cell, and the second portion comprises a second axially extending column of the outer frame including a second strut cell that is adjacent to the first strut cell, and the at least one rotation joint couples a first strut of the first strut cell with a second strut of the second strut cell that is non-integral with the first strut.

16. The prosthetic heart valve of claim 10, wherein the prosthetic heart valve includes one or more distal anchors adapted to hook over one or more native leaflets of the defective native heart valve to anchor the prosthetic heart valve to the defective native heart valve.

17. A prosthetic heart valve for replacing the function of a defective native heart valve, the prosthetic heart valve comprising:
a tubular metallic frame surrounding a flow channel; and
a plurality of prosthetic valve leaflets positioned within the flow channel;
wherein a plurality of longitudinal hinges is provided in a wall of the frame such that at least a first region of the frame is capable of pivoting relative to a second region of the frame.

18. The prosthetic heart valve of claim 17, wherein the frame is made from a shape memory material.

19. The prosthetic heart valve of claim 17, wherein the frame is made from stainless steel.

20. The prosthetic heart valve of claim 17, wherein the frame is sized for replacement of a native mitral or tricuspid heart valve.
